(19) United States
(12) Patent Application Publication
(10) Pub. No.: US 2003/0032774 A1
(43) Pub. Date: Feb. 13, 2003

(54) Novel opioid peptides for the treatment of pain

(75) Inventors: William Brown, Laval (CA); John DiMaio, Montreal (CA); Peter Schiller, Montreal (CA); Rene Martel, Saint Laurent (CA); Francis Martel, St-Philippe (CA); Diane Martel, Ile des Soeurs (CA); Pierre Martel, Pointe-Claire (CA)

Correspondence Address: PILLSBURY WINTHROP, LLP, P.O. BOX 10500, MCLEAN, VA, 22102, US

(73) Assignee: AstraZeneca AB

(21) Appl. No.: 09/994,078

(22) Filed: Nov. 26, 2001

Related U.S. Application Data
(62) Division of application No. 09/159,518, filed on Sep. 24, 1998, now abandoned.
Application No. 09/159,518 is a continuation-in-part of application No. 08/392,918, filed on Mar. 3, 1995, now abandoned.
Application No. 09/159,518 is a continuation-in-part of application No. 08/718,585, filed on Oct. 2, 1996, now Pat. No. 6,337,319.

(30) Foreign Application Priority Data
Feb. 21, 1994 (GB) 9403263.8
Apr. 25, 1994 (GB) 9408179.1
May 3, 1994 (SE) 9401519-5
Aug. 18, 1995 (SE) 9502877-5
Nov. 7, 1995 (SE) 9503924-4
Feb. 15, 1995 PCT/SE95/00158
Aug. 14, 1996 PCT/SE96/01011

(51) Int. Cl. C07K 5/10; A61K 38/06

(52) U.S. Cl. 530/330; 514/18

(57) ABSTRACT

This invention relates to opioid peptides, to pharmaceutical compositions containing the peptides, and to methods for controlling pain by administering the peptides or pharmaceutical compositions. The peptides have a high degree of selectivity for the μ-opioid receptor. Although they are highly lipophilic, the peptides do not readily cross the blood brain barrier (BBB). Because the peptides act peripherally, they substantially avoid the side effects associated with central analgesic action.

### FIELD OF THE INVENTION

The present invention is directed to opioid-like peptide compounds that exhibit peripheral analgesic activity and selectivity for the μ subtype of opioid receptors.

### BACKGROUND OF THE INVENTION

The development of new and effective agents for alleviating the peripheral pain of patients is a major objective of medicine. In this regard, it has recently been reported that opiates have significant peripheral analgesic activity (Barber et al., Med. Res. Rev. 12:525 (1992); and Stein, Anesth. Analg. 76:182(1993)). Quatemary salts of known centrally acting opioid alkaloids have been used as pharmacological probes to distinguish between peripheral and central analgesic responses. These quaternary salts have a permanent positive charge and show restricted penetration of the blood-brain barrier (see Smith, et al., Life Sci. 31:1205 (1982); Smith, et al., Int. J. Tiss. Reac. 7:61 (1985); Lorenzetti, et al., Braz. J. Med. Biol. Res. 15:285 (1982); Brown, et al., Neuropharmacol., 24:181 (1985); Bianchi et al., Life Sci. 30:1875 (1982); and Russel, et al., Eur. J. Pharmacol., 78:255 (1982)).

Many endogenous peptides of mammalian and amphibian origin (e.g. the endorphins) bind to opioid receptors and elicit an analgesic response similar to classic narcotic opiates. This led to the hope that these peptides might be produced commercially and administered to patients to relieve pain. Unfortunately, administration of opioid peptides to mammals has been shown to affect the central nervous system. For example, it has been reported that TAPP(H-Tyr-D-Ala-Phe-Phe-NH₂) exhibits antinociceptive properties both peripherally and centrally (Schiller, et al., Proceedings of the 20th European Peptide Symposium (1988)). Because side effects such as respiratory depression, tolerance, physical dependence capacity, and precipitated withdrawal syndrome are caused by nonspecific interactions between opiates and central nervous system receptors (see Budd, in: International Encyclopedia of Pharmacology and Therapeutics, Williams and Wilkinson, eds., Pergammon: (Oxford), 112, p. 51 (1983)), scientists have attempted to structurally modify opioid peptides to lessen CNS penetration. Highly polar analogues of enkephalins and dermorphins have been prepared which retain high antinociceptive activity but show limited central nervous system penetration (see, Follenfant, et al., Br. J. Pharmacol. 93:85 (1988); Hardy, et al., J. Med. Chem., 32:1108 (1989)).

In order to be effective, an analgesic agent must act at the particular opioid receptors responsible for providing pain relief. Three different types of opioid receptors have been identified. The first type, δ, exhibits a preference for enkephalin-like peptides. The second, μ, shows enhanced selectivity for morphine and other polycyclic alkaloids. Finally, κ-type receptors exhibit equal affinity for either group of ligands and preferential affinity for dynorphin. In general, it appears that the μ receptors are the ones primarily responsible for mediating analgesic effects, although the δ- and the κ-receptors may also play a role.

Each opioid receptor, when bound by opiate, causes a unique biological response. When an opiate activates more than one receptor type, the biological response associated with each is induced, thereby producing side effects. Thus, an ideal peptide for alleviating pain would both act specifically on μ-type opioid receptors and not cross the blood-brain barrier. The present invention is directed to peptides that approach this ideal.

### SUMMARY OF THE INVENTION

The present invention is directed to peptides that exhibit both specificity and selectivity for binding to μ-opioid receptors. As used herein, a peptide has specificity for the μ-type receptor if it has a greater affinity (i.e. lower binding inhibition constant, Kᵢ) for this receptor than for either δ- or κ-type receptors. The term “selectivity” refers to the ability of an opiate or opioid peptide to discriminate among several opioid receptors and to bind to only one. The selectivity of an opioid peptide is indicated by the ratio of binding inhibition constants. For instance, the ratio of binding inhibition constants, Kᵢδ/Kᵢμ, is a value that may be used to measure selectivity. This ratio represents the relationship of the affinities for binding to the μ and δ receptors. A higher value for this ratio indicates a greater preference of ligand to bind with the μ receptor over the δ receptor. One conventional opioid peptide analog, H-Tyr-D-Ala-Gly-Phe(NMe)-Gly-ol (DAGO), is known to be one of the most μ selective opioid peptide analogues. This peptide shows a Kᵢδ/Kᵢμ value of 1050. Leu-enkephalin, on the other hand, shows a Kᵢδ/Kᵢμ value of 0.2. This fractional value reflects a pronounced affinity for the δ receptor over the μ receptor.

The invention also provides for peptides which primarily interact with opioid receptors on peripheral nerve terminals. Surprisingly, even though such peptides are lipophilic, they do not substantially cross the blood-brain barrier. The combination of ×-receptor selectivity and binding localized in the periphery are indications that the peptides have fewer and less severe side effects as compared to those associated with previously reported opiates and opioid peptides.

A. Peptides with Analgesic Effects

In its first aspect, the compounds of the present invention are represented by formula (A):

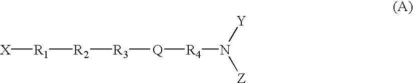

with peripheral analgesic effect, wherein: X is selected from the group consisting of H and C₁₋₆ alkyl; Y and Z are independently selected from the group consisting of H, cyclic aralkyl, and C₁₋₆ alkyl; R₁ is a tyrosyl residue or a 2′,6′-dimethyltyrosyl residue; R₂ is an amino acid having the R-configuration, aminoisobutyric acid, cyclopropylalanine, cyclohomoleucine or cycloleucine; R₃ is an aromatic amino acid; R₄ is an aromatic amino acid residue; Q is an amide bond or an interposed amide bond mimetic; with the following provisos: a) when R1 is a tyrosyl residue; R₂ is D-alanine;X, Y, and Z are H; and R₃ is phenylalanine; then R₄ is not unsubstituted phenylalanine or phenylalanine substituted with 4NO₂ or 4N₃; b) when R1 is a tyrosyl residue; R2 is D-alanine; X, Y, and Z are H; and R₄ is phenylalanine; then R₃ is not unsubstituted phenylalanine or phenylalanine substituted with 4NO₂; c) when R₁ is a tyrosyl residue; R₂ is D-alanine; X, Y, and Z are H; and R₄ is 1′-naphthylalanine; then R₃ is not 1′-naphthylalanine or 2′-naphthylalanine; d) when R₁ is a tyrosyl residue; R₂ is D-alanine; and X, Y and Z are H; then R₃ and R₄ are not tryptophan; e) when R₁ is a tyrosyl residue; R₂ is a D-amino acid with a lower alkyl or lower thioalkyl group as a side chain; and R₄ is a neutral amino acid; then R₃ is not unsubstituted phenylalanine; and wherein said compound is not selected from the group consisting of: H-Tyr-D-Phe-Phe-Phe-NH₂; H-Tyr-D-NMePhe-Phe-Phe-NH₂; H-Tyr-D-Tic-Phe-Phe-NH₂; H-Tyr-Pro-Phe-Thr(Bz1)-NH2; H-Tyr-Pro-Phe-Phe-NH₂; H-Tyr-Pro-Phe-Apb-NH₂; H-Tyr-Pro-Phe-App-NH₂; H-Tyr-Pro-Phe-Aph-NH₂; and H-Tyr-Pro-Apb-Phe-NH₂; wherein Apb is 2-amino4-phenylbutanoic acid, App is 2-amino-5-phenyl pentanoic acid and Aph is 2-amino-6-phenylhexanoic acid.

The invention also provides for pharmaceutically acceptable compositions comprising the peptides and for methods of relieving pain by administering such compositions.

B. Pentapeptides with Phe(pF) in the Third Position

In a second aspect, the present invention is directed to peptides of formula (A) with Phe(pF) in the R₃ position and in which Q is a peptide bond. These peptides may be represented by the formula:

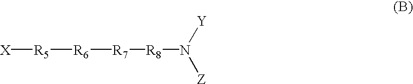

and salts, derivatives and analogues thereof wherein: R₅ is Tyr, 2′,6′-dimethyltyrosine, or an analog or derivative thereof; R₆ is D-Ala or D-Arg; R₇ is Phe(pF); R₈ is Phe or Phe(pF); X is H or C1-6 alkyl; and Y and Z are independently H, aralkyl or C₁₋₆, alkyl.

The peptides may be used in pharmaceutical compositions in admixture with a pharmaceutically acceptable carrier and/or a second therapeutically active agent. These compositions may be administered to mammal as means for relieving pain.

### BRIEF DESCRIPTION OF THE TABLES AND FIGURES

Table 1 lists in vivo and in vitro activity of hydrophobic dermorphin related tetrapeptides.

FIGS. 1A-1D indicate the time course of the analgesic effect of morphine (10 mg/kg) (FIG. 1A) and exemplary test compounds (FIG. 1B: BCH2463; FIG. 1C: BCH2462; FIG. 1D: BCH2687)

FIG. 2 shows dose response curves for BCH2463 in the phenylquinone-induced writhing assay (in the mouse sc.) ED₅₀=0.5 mg/kg at 20 minutes post administration.

FIG. 3 lists comparative analgesic time course of BCH1774 and BCH2463 in the phenylquinone-induced writhing assay (mouse s.c.)

FIGS. 4A, 4B, 5A, 5B, 7A, and 7B illustrate the inhibitory effect of compounds of formula (B) in two different hot-plate assays.

FIG. 6 illustrates the inhibitory effect of H-Tyr-D-Ala-Phe-Phe-NH2 in a hot-plate assay.

FIGS. 8A and 8B illustrate the inhibitory effect of compounds of formula (B) in the tail-flick assay.

### DEFINITIONS AND ABBREVIATIONS

The following common abbreviations are used throughout the specification and in the claims:

| | |
|---|---|
| Abu - aminobutyric acid | Aib - aminoisobutyric acid |
| Ala - alanine | Arg - arginine |
| BOP-benzotriazolyl-N-oxy-tris(dimethylaniino) | Chl - cyclohomoleucine |
| phosphordumhexafluorophosphate | |
| Cle - cycloleucine | Cys (Bzl) - cysteine (benzyl) |
| DMF-dimethylformamide | Dmt - 2′6′-dimethyltyrosyl |
| FMOC -9-fluorenylmethyloxycarbonyl | Gln - glutamine |
| Glu - glutamic acid | Gly - glycine |
| GPI - guinea pig ileum | His - histidine |
| Hph - homophenylalanine | HOBT - N-hydroxybenzothiazole |
| Ile - isoleucine | Leu - leucine |
| Met - methionine | MVD - mouse vas deferens |
| Nal-1′-, or 2′-naphthylalanine | Nle - norleucine |
| Nva - norvaline | PBQ -phenyl-ρ-benzoquinone |
| pE - parafluoro | Phe - phenylalanine |
| Phe(pF) - parafluoro phenylalanine | Phg - phenylglycine |
| Pmc-2,2,5,7,8 pentamethylchroman-6-sulfonyl | Pro - proline |
| Ser - serine | TAPP - H-Tyr-D-Ala-Phe-Phe-NH₂ |
| tBU-tert-butyl | TFA-trifluoroacetic acid |
| Thr - threonine | Tic - tetrahydroisoquinoline-3- |
| | carboxylic acid |
| Trp - tryptophan | TSPP - H-Tyr-D-Ser-Phe-Phe-NH₂ |
| Tyr - tyrosine | ∇ - cyclopropyl |

The terms “amino acid,” and “aromatic amino acid,” as used herein, include naturally occurring amino acids as well as non-natural amino acids, their derivatives, and analogues, commonly utilized by those skilled in the art of chemical synthesis and peptide chemistry. Also analogues of TAPP in which the phenylalanine is para-substituted at position 4 with a nitro or azido residue are included. A list of non-natural and non-proteogenic amino acids may be found in “The Peptide”, vol 5, 1983, Academic Press, Chapter 6 by D.C. Roberts and F. Vellaccio which is incorporated herein by reference. Examples of aromatic amino acids include tyrosine, tryptophan, phenylglycine, histidine, naphthylalanine, tetrahydroisoquinline-3carboxylic acid and benzylcysteine. Other examples of aromatic amino acids include phenylalanine substituted on its aromatic ring with, for example, CH₃, C₂H₅, F, Cl, Br, NO₂, OH, SH, CF₃, CN, COOH, and CH₂COOH. These examples are intended to be exemplary only and are not intended to limit the invention in any way.

The term “ED₅₀” as shown in Table 1 for the PBQ writhing assays is defined as the dose of drug which induces a 50% reduction in the number of writhes observed compared to the control. The term “ED₅₀” used in hotplate assays is defined as the dose of drug required to increase the latency of response 2-fold compared to controls and was determined by parallel-line probit analysis.

The term “interposed amide bond mimetic” is a bond in which the carbonyl group and the NH group of an amide bond are interchanged.

The term “Kᵢ” is the binding inhibition constants The term “Kᵢδ/Kᵢμ” is a value used to indicate selectivity. This ratio represents the relationship of the affinities of opioid peptides for binding to the μ and δ-receptors.

The term “R-configuration” refers to the three dimensional arrangement of substituents around a chiral element. A general system for designating absolute configuration is based upon a priority system which is well-known to persons skilled in the art and is briefly described hereafter. Each group attached to the chiral center is assigned a number according to priority. The molecule is viewed from the side opposite the lowest priority. The configuration is specified “R” if the eye proceeds in a clockwise direction when traveling from the group of highest priority to the group of lowest priority.

The term “residue” when applied to an amino acid, means a radical derived from the corresponding amino acid by removing the hydroxyl of the carboxyl group and one hydrogen from the amino group.

### DETAILED DESCRIPTION OF THE INVENTION

A. Compounds of Formula (A)

In its first aspect, the compounds of the present invention are defined by formula (A) and the associated definition of terms set forth in the Summary section above. For these compounds, as well as for each group of preferred compounds discussed below, there is a preferred subgroup in which X is H.

One group of preferred compounds is represented by formula (A) and derivatives and analogues thereof, wherein R₂ is an amino acid residue having the R-configuration, aminoisobutyric acid, cyclopropylalanine, cyclohomoleucine or cycloleucine; with the proviso that when R₁ is a tyrosyl residue; R₂ is a D-alanine; and Y, and Z are H; then R₃ and R₄ are different and are selected from the group consisting of phenylalanine and tryptophan.

Another group of preferred compounds is represented by formula (A) and derivatives and analogues thereof, wherein Q is an amide bond or an interposed amide bond mimetic of the formula Q₁-Q₂ wherein Q₁, is selected from the group consisting of CH₂, CHOH, C═O, C═S, and CH═, and Q is selected from the group consisting of CH₂, NH, S, SO, SO₂, O and CH═ with the proviso that when Q₁, is CH═, then Q₂ is CH═.

Further preferred compounds are represented by formula (A) and derivatives and analogues thereof, wherein, Y and Z are H; R₃ and R₄ are independently an aromatic amino acid; and R₂ is an amino acid having the R-configuration, aminoisobutyric acid, cyclopropylalanine, cyclohomoleucine or cycloleucine, with the proviso that when R₁ is a tyrosyl residue, and R₂ is D-alanine, then R₃ and R₄ are different and are selected from the group consisting of phenylalanine and tryptophan. In one subset of these preferred compounds, Q is an amide bond or an interposed amide bond mimetic of the formula Q₁-Q₂ wherein Q₁, is selected from the group consisting of CH₂, CHOH, C═O, C═S, and CH═, and Q₂ is selected from the group consisting of CH₂, NH, S, SO, SO₂, O and CH═ with the proviso that when Q₁, is CH═, then Q₂ is CH═.

Other preferred compounds are represented by formula (A) and derivatives and analogues thereof, wherein, R₂ is an amino acid having the R-configuration, aminoisobutyric acid, cyclopropylalanine, cyclohomoleucine or cycloleucine with the proviso that R₂ is not D-alanine; and R₃ and R₄ are phenylalanyl residues.

Still, other preferred compounds are represented by formula (A) and derivatives and analogues thereof, wherein, R₁ is a tyrosyl residue; R₂ is selected from the group consisting of D-norvaline, D-serine, and D-arginine; R₃ and R₄ are phenylalanyl residues; and Q is a peptide bond.

Another group of preferred compounds is represented by formula (A) and derivatives and analogues thereof, wherein, Y and Z are independently selected from the group consisting of H, aralkyl, and C₁₋₆ alkyl; R₁ is a tyrosyl residue, 2′,6′-dimethyltyrosyl residue, or an analogue or derivative thereof; R₃ is an aromatic acid; R₄ is an aromatic amino acid; and R₂ is an amino acid residue having the R-configuration; with the proviso that when R₂ is D-alanine, R₁ is a tyrosyl residue, and Y and Z are H, then R₃ and R₄ are independently selected from the group consisting of phenylalanine, and tryptophan, but are not the same, Q is an amide bond or an interposed amide bond mimetic of the formula Q₁-Q₂ wherein Q, is selected from the group consisting of CH₂, CHOH, C═O, C═S, and CH═, and Q₂ is selected from the group consisting of CH₂, NH, S, SO, SO₂, O, and CH— with the proviso that Q, is CH═, then Q₂ is CH═.

Additional preferred compounds are represented by formula (A) and derivatives and analogues thereof, wherein, Y and Z are H; R1 is a tyrosyl residue, a 2′, 6′-dimethyltyrosyl residue, or an analogue or derivative thereof; R₃ and R₄ are independently an aromatic amino acid; R₂ is an amino acid having the R-configuration; with the proviso that when R₂ is D-alanine, and R₁ is a tyrosyl residue, then R₃ and R₄ are independently selected from the group consisting of phenylalanine and tryptophan, but are not the same; Q is an amide bond or an interposed amide bond mimetic of the formula Q₁-Q₂ wherein Q₁ is selected from the group consisting of CH₂, CHOH, C═O, C═S, and CH═, and Q₂ is selected from the group consisting of CH₂. NH, S, SO, SO₂, O, and CH═, with the proviso that when Q₁ is CH═, then Q₂ is CH═.

Another group of preferred compounds is represented by formula (A) and derivatives and analogues thereof, wherein, Y and Z are H; R₁ is a tyrosyl residue, 2′,6′-dimethyltyrosyl residue, or an analogue or derivative thereof; R₂ is an amino acid having the R-configuration with the proviso that R₂ is not D-alanine; R₃ and R₄ are phenylalanyl residues; Q is an amide bond or an amide bond mimetic of the formula Q₁-Q₂ wherein Q₁ is selected from the group consisting of CH₂, CHOH, C═O, C═S, and CH═, and Q₂ is selected from the group consisting of CH₂, NH, S, SO, SO₂, O, and CH═, with the proviso that when Q₁ is CH═, then Q is CH═.

The most preferred compounds are represented by formula (A) and derivatives and analogues thereof, wherein, Y and Z are H; R₁ is a tyrosyl residue; R₂ is selected from the group consisting of D-norvaline, D-serine, and D-arginine; R₃ and R₄ are phenylalanyl residues, and Q is a peptide bond.

Specific, individual, preferred compounds of this invention are as follows:

H-Tyr-Aib-Phe-Phe-NH₂;

H-Tyr-D-Nle-Phe-Phe-NH₂;

H-Tyr-D-Ala-Phe-2′-Nal-NH₂;

H-Tyr-D-Ala-D-Phe-Phe-NH₂;

H-Tyr-D-Ala-Phe(4NO₂)-Phe(4NO₂)—NH₂;

H-Tyr-D-Ala-Phe-Tic-NH₂;

H-Tyr-D-Ala-Phe-Phe(NMe)-NH₂;

H-Tyr-D-Ala-Phe-1′-Nal-NH₂;

H-Tyr-D-Ala-Trp-Phe-NH₂;

H-Tyr-D-Ala-Phe-Trp-NH₂;

H-Tyr-VAla-Phe-Phe-NH₂;

VCH₂-Tyr-D-Ala-Phe-Phe-NH₂;

H-Tyr-D-Nle-Phe-Trp-NH₂;

H-Tyr-D-Nle-Phe-2′-Nal-NH₂;

H-Tyr-D-Nle-Trp-Phe-NH₂;

H-Tyr-D-Ala-Trp-2′-Nal-NH₂;

H-Tyr-D-Nle-Trp-2′-Nal-NH₂;

H-Tyr-D-Nle-Trp-Trp-NH₂;

H-Tyr-D-Nva-Phe-Phe-NH₂;

H-Tyr-D-Ser-Phe-Phe-NH₂;

H-Tyr-D-Val-Phe-Phe-NH₂;

H-Tyr-D-Leu-Phe-Phe-NH₂;

H-Tyr-D-Ile-Phe-Phe-NH₂;

H-Tyr-D-Abu-Phe-Phe-NH₂;

H-Tyr-Chl-Phe-Phe-NH₂;

H-Tyr-Cle-Phe-Phe-NH₂;

H-Tyr-D-Arg-Phe-Phe-NH₂;

H-Tyr-D-Cys-Phe-Phe-NH₂;

H-Tyr-D-Thr-Phe-Phe-NH₂;

H-DMT-D-Ser-Phe-Phe-NH₂;

H-Tyr-D-Ala-Phe-Phe-OH trifluoroacetate;

H-Tyr-D-Ala-Phe-Phg-NH₂ trifluoroacetic acid salt;

H-Tyr-D-Arg-Phe-Hph-NH₂ bis-trifluoroacetic acid;

H-DMT-D-Ala-Phe-Phe-NH₂ trifluoroacetic acid;

H-D-DMT-D-Ala-Phe-Phe-NH₂ trifluoroacetic acid salt;

H-Tyr-D-Ala-Phe-Hph-NH₂ trifluoroacetic acid salt;

H-Tyr-D-Ala-Phe-Cys(Bzl)-NH₂ trifluoroacetic acid salt;

H-Tyr-D-Arg-Hph-Phe-NH₂ bis-trifluoroacetic acid salt;

H-Tyr-D-Arg-Phg-Phe-NH₂ bis-trifluoro acetic acid salt;

H-Tyr-D-Ala-Phe-Phe-CH₂OH hydrochloride salt;

H-Tyr-D-Ala-Hph-Phe-NH₂ trifluoroacetic acid salt;

H-Tyr-D-Met-Phe-Phe-NH₂ trifluoroacetic acid salt;

H-Tyr-D-Arg-Phe-D-Phe-NH₂ bis-trifluoroacetic acid salt;

H-Tyr-D-Ala-Phg-Phe-NH₂ trifluoroacetic acid salt;

H-Tyr-D-Ala-Phg-Phe-NH₂ trifluoroacetic acid salt;

H-Tyr-D-Arg-Phe-D-Phe-NH₂ bis-trifluoroacetic acid salt;

H-Tyr-D-Arg-Phe-D-Phe(pF)-NH₂ ditrifluoroacetic acid salt;

H-Tyr-D-Ala-Phe-Phe(pF)-NH₂ trifluoroacetic acid salt; and

H-Tyr-D-Ala-Phe-D-Phe(pF)-NH₂ trifluoroacetic acid salt.

The most preferred compounds for use in pharmaceutical compositions and in methods of treating patients for pain are: H-Tyr-D-Nva-Phe-Phe-NH₂; H-Tyr-D-Ser-Phe-Phe-NH₂; and H-Tyr-D-Arg-Phe-Phe-NH₂. Of these, the most highly preferred is H-Tyr-D-Arg-Phe-Phe-NH₂.

B. Analgesic Activity of the Compounds of Formula A

A number of tetrapeptides based on formula (A) have been prepared and evaluated as opioid receptor ligands and systemically acting analgesic agents. These compounds are listed in Table 1 along with their respective binding inhibition constants and receptor selectivity ratios.

2′,6′-dimethyltyrosine (Dmt) may be substituted for tyrosine in the opioid peptide compounds. Experiments have shown that the substitution of Dmt for tyrosine at the R₁ position, the first amino acid residue in formula (A), enhances the potency of the opioid peptide at the μ receptor by up to two orders of magnitude. Selectivity for the μ-receptor increases when the compound includes Dmt at the R₁ position. This substitution causes a corresponding shift in the ratio of binding inhibition constants to reflect the increased 1-receptor selectivity.

Many of the compounds listed in Table 1 show good p-receptor binding but weak analgesic effect in the mouse writhing assay. This anomaly may be due to rapid proteolysis, rapid clearance, or both. For example, when the prototype lipophilic dermorphin peptide TAPP (BCH1774) was exposed to brushborder kidney membranes, it was observed to be degraded within 15-30 minutes. Of the peptides listed in Table 1, three preferred compounds, other than TAPP itself, exhibit an increased analgesic effect in vivo. These three compounds are H-Tyr-D-Nva-Phe-Phe-NH₂ (BCH2462), H-Tyr-D-Ser-Phe-Phe-NH₂ (13CH2463), and H-Tyr-D-Arg-Phe-Phe-NH₂ (BCH2687). BCH2462, BCH2463, and BCH2687 have been shown to exhibit peripheral analgesia. No central analgesic effect was observed using these peptides even at doses of 100 mg/kg in the mouse hot plate test.

As shown in Table 1, the ED₅₀ value for TAPP (BCH1774) is 1.4. The corresponding values for H-Tyr-D-Nva-Phe-Phe-NH₂ (B CH2462), and H-Tyr-D-Ser-Phe-Phe-NH₂ (BCH2463), and H-Tyr-D-Arg-Phe-Phe-NH₂ (BCH2687) are 2.7, 0.5, and 0.5 respectively. The ED₅₀ values for the remaining compounds in Table 1 are higher than these figures. Although the ED₅₀ value of BCH2813 was only 0.15, it was found to act centrally at doses of about 40 mg/kg in the hot plate test.

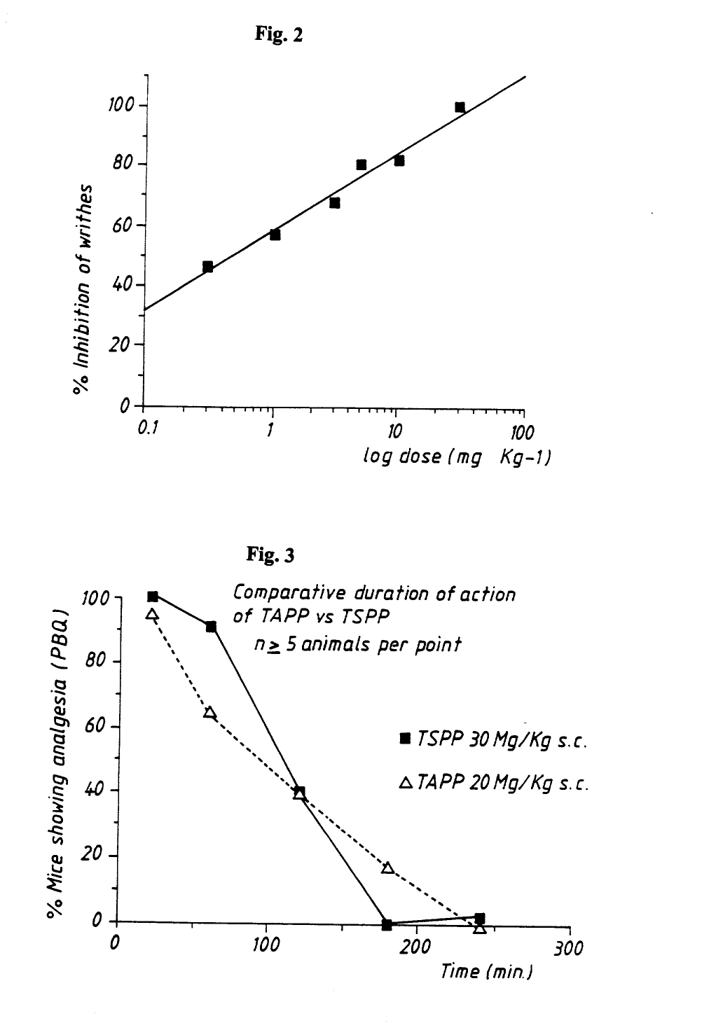

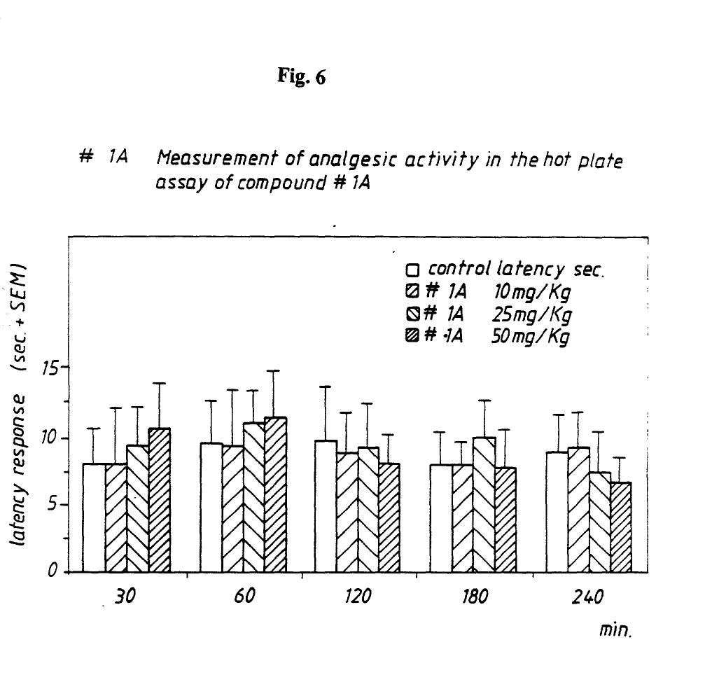

These results indicate that the compounds BCH1774, BCH2462, and BCH2463 still undergo proteolysis but they have a longer half life and therefore are more effective as analgesic agents. In FIG. 6, the duration in vivo of analgesic effects caused by BCH1774 (TAPP) and BCH2463 (TSPP) were compared. Using 30 mg/kg s.c. of BCH2463 and 20 mg/kg s.c. of BCH1774, FIG. 3 indicates that the analgesic effect of BCH1774 lasted longer than for BCH2463, possibly indicating a slightly accelerated in vivo proteolysis of BCH2463 than for BCH1774.

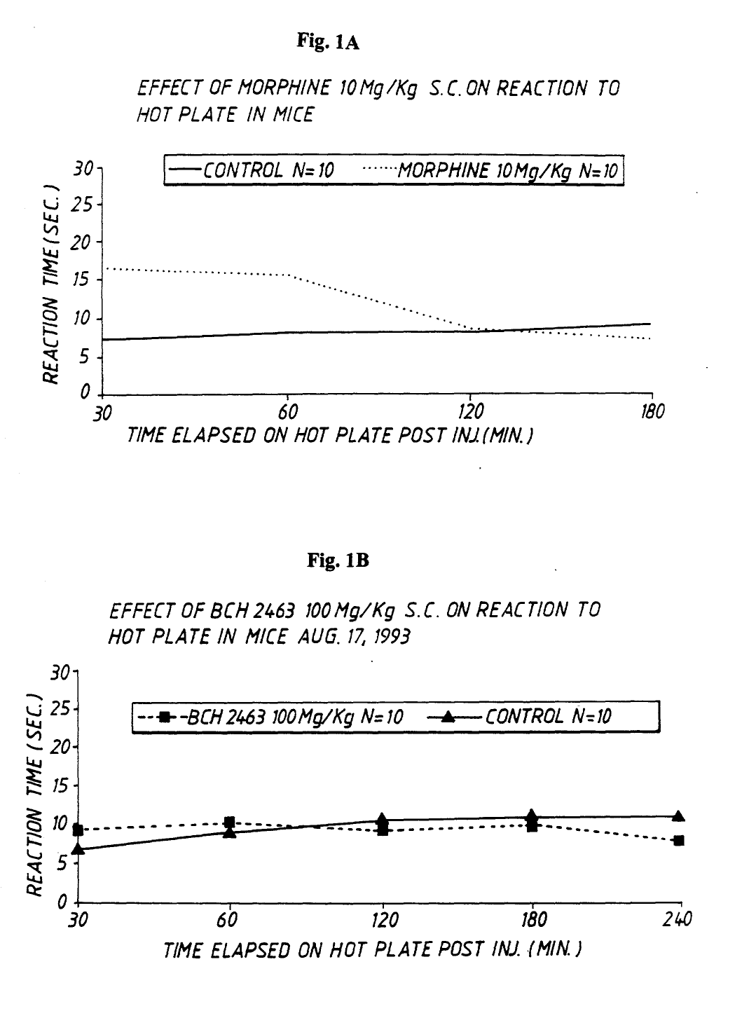

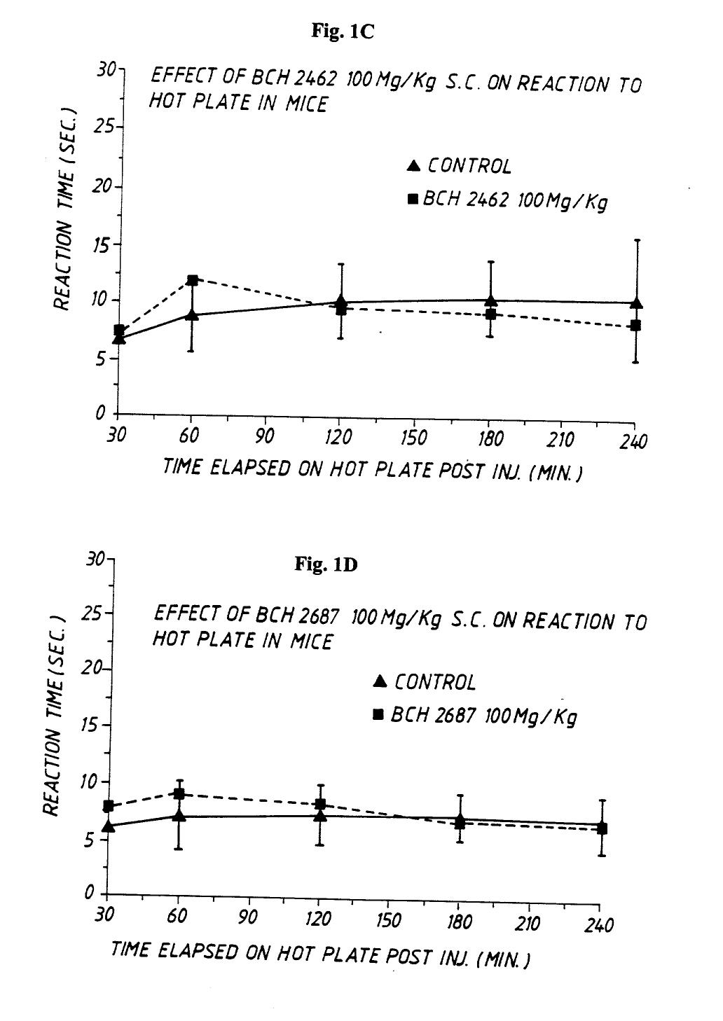

FIGS. 1A-D show the effects of morphine, BCH2463 (TSPP), BCH2462 (TNPP) and BCH2687 determined by evaluating the reaction of the mice in the hot plate test. As shown in FIG. 1A, the reaction time of the mice treated with 10 mg/kg of morphine is approximately 17 seconds. The reaction time of the mice treated with 100 mg/kg of BCH2463 (FIG. 1B) is about 9 seconds compared to a control value of approximately 7 seconds. These results indicate that while morphine inhibits the nociceptive thermal stimulus, BCH2463 does not; but BCH2463 is a potent analgesic agent as is shown by the inhibition of chemically-induced writing (FIG. 2). The reaction time of the mice treated with BCH2462 and with BCH2687 (FIG. 1C and FIG. 1D) is approximately 8 seconds.

The effects of inhibition of proteolytic metabolism of BCH2463 by the inhibitor DL-thiorphan has been studied and also the metabolic breakdown of BCH2463 mediated by brush border kidney membranes. The data obtained indicate that the kidney may be the principal site of clearance and metabolism for the compound BCH2463. From FIG. 2, it appears that the endopeptidase enzyme EC24-11, which is inhibited by DL-thiorphan, is the preliminary mediator of BCH2463 proteolysis by brush border kidney extract.

Both BCH1774 (TAPP) and BCH2462 (TNPP) exhibited lethal effects upon mice when administered at 1-5 mg kg.⁻¹ i.v. bolus dose of drug. In contrast, BCH2463 (TSPP) surprisingly did not exhibit any lethal effects at doses up to 20 mg/kg i.v. In addition, peptides were safe when administered subcutaneously (s.c.) at doses greater than 100 mg/kg. Thus, the structural paradigm exemplified by BCH1774 can be modified while maintaining exclusion from the central nervous system even at doses as high as 100 mg kg⁻¹ s.c. and toxicity can be minimized.

C. Compounds of Formula B

In a second aspect, the compounds of the present invention are defined as set forth by formula B and the associated definition of terms as set forth in the Summary section above. Compounds of the invention include but are not limited to:

| | |
|---|---|
| Compound #1B | H-Tyr-D-Ala-Phe(pF)-Phe(pF)-NH₂; |
| Compound #1C | H-Tyr-D-Ala-Phe(pF)-Phe-NH₂; |
| Compound #2B | H-Tyr-D-Arg-Phe(pF)-Phe(pF)-NH₂; and |
| Compound #2C | H-Tyr-D-Arg-Phe(pF)-Phe-NH₂. |

In a preferred embodiment, the compounds of the invention are selected from the group consisting of:

| | |
|---|---|
| Compound #1C | H-Tyr-D-Ala-Phe(pF)-Phe-NH₂; and |
| Compound #2C | H-Tyr-D-Arg-Phe(pF)-Phe-NH₂. |

In the most preferred embodiment, the compound of the invention is

| | |
|---|---|
| Compound #IC | H-Tyr-D-Ala-Phe(pF)-Phe-NH₂. |

As with the compounds of formula (A), the amino acid derivative 2′,6′-dimethyltyrosine (Dmt) may be substituted for tyrosine in the opioid peptide compounds of formula B.

D. Analgesic Activity of the Compounds of Formula B

PBQ (phenyl-ρ-benzoquinone) induced writhing in mice is an assessment of both central and peripheral analgesia (see Sigmund, et al., Proc. Soc. Exp. Biol. Med. 95:729 (1957)). Central analgesia is determined by the inhibition of a hot-plate response in mice (see, Wolfe, et al., J. Pharmacol. Exp. Ther. 80:300 (1944)). Assays measuring opioid receptor binding affinities for μ and δ receptors as well as the GPI assay were determined through experimental protocol set out in Schiller et al. (Biophys. Res. Commun. 85:1322 (1975)). The references by Sigmund, et al., Wolfe, et al., and Schiller, et al. are each hereby incorporated by reference.

Comparison of the activities of compounds of the invention in the GPI, writhing, hot-plate, and tail flick assays demonstrated that the analgesic effects were predominantly mediated in the periphery. Peripheral analgesia was shown by a high potency in the writhing test coupled with a low potency in the hot-plate test or the tail flick test.

E. Synthesis of Compounds of Formula A and B

The compounds of the present invention can be produced by methods well known in the art of peptide chemistry see, e.g, Principle of Peptide Synthesis, Bodansky M., Spinger-Verlag, New York, (1984); The Peptides, Analysis, Synthesis, Biology, Gross and Meienhofer eds, Academic Press (1979)). Synthesis was performed using a solid phase procedure as described in the Examples section.

Commercially available parafluoro-phenylalanine (Phe(pF)) was employed at the appropriate step of synthesis in Examples 5 and 6. 2′,6′-dimethyltyrosine may also be incorporated in the synthesis and is prepared according to established chemical synthesis techniques.

Pharmaceutically acceptable salts of the peptides of this invention may be formed by reaction with an appropriate acid. Suitable acid addition salts may be formed by the addition of acids such as hydrochloric, hydrobromic, phosphoric, acetic, fumaric, salicylic, citric, lactic, mandelic, tartaric, oxalic, methanesulphonic, and other suitable acids known to persons skilled in the art.

F. Pharmaceutical Compositions

The present invention also provides for pharmaceutical compositions. Suitable compositions have a pharmaceutically effective amount of one or more peptides of the invention, or pharmaceutically acceptable salts thereof, in admixture with a pharmaceutically acceptable carrier or adjuvant. Formulations may be prepared using methods that are standard in the art (see e.g., Remington's Pharmaceutical Sciences, 16th ed., A. Oslo Editor, Easton Pa. (1980)). Specific dosage forms include tablets, pills, capsules, powders, aerosols, suppositories, skin patches, parenterals, and oral liquids including oil aqueous suspensions, solutions and emulsions. Sustained release dosage forms may also be used.

The peptides may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations. Solutions can be prepared using water or physiological compatible organic solvents such as ethanol, 1,2-propylene glycol, polyglycols, dimethyl sulfoxide, fatty alcohols, triglycerides, partial esters of glycerine and the like. Parenteral compositions may be prepared using conventional techniques and include sterile isotonic saline, water, 1,3-butanediol, ethanol, 1,2-propylene glycol, polyglycols mixed with water, Ringer's solution, etc

G. Method of Treating of Pain

The present invention also provides for a method of treatment of pain in mammals, including humans. The method comprises administering a pharmaceutically effective amount of TAPP, a peptide of formula A, a peptide of formula B, or a pharmaceutically acceptable salt or composition thereof to a mammal in need of pain relief The peptides can be administered to a human patient in a dosage of about 0.01 to 100 mg/kg, more preferably at about 0.05 to 20 mg/kg, and most preferably at about 0.1-1 mg/kg.

Any route of administration and dosage form is compatible with the treatment method and a peptide may be administered as either the sole active agent or in combination with other drugs. Preparations may be administered epidurally, orally, perorally, internally, rectally, nasally, vaginally, lingually, sublingually, transdermally, intravenously, intraarterially, intramuscularly, intraperitoneally, intracutaneously and subcutaneously.

### EXAMPLES

### Example 1

### Solid Phase Peptide Syntheses of Opioid Peptides of Formula A

Synthetic peptides were prepared using “RINK” resin, 4-(2′,4′-dimethoxy-phenyl-Fmoc-aminomethyl)-phenoxy resin (Novabiochem or Advanced Chemtech) and the relevant C-terminal Na-Fmoc-L-Amino acid residue of each peptide to be synthesized.

All L- and D-amino acids had their alpha group Fmoc-protected (9-fluorenyl-methyloxycarbonyl) and the following side chain protection groups: t-butyl ether (tBu) for serine, threonine and tyrosine; t-butyl ester (OtBu) for aspartic acid and glutamic; t-butyloxycarbonyl (tboc) for lysine and 2,2,5,7,8-pentamethylchroman-6-sulphonyl (pmc) for arginine and trityl (trt) for cysteine.

Dimethylformamide (Anachemia, dimethylamine-free purity) was treated with activated 4 Å molecular sieves. Piperidine (Advanced Chemtech) was used without further purification. DCC (dicyclohexylcarbodiimide) and HOBt (hydroxybenzotriazole) were obtained from Fluka and Advanced Chemtech respectively.

Solid phase peptide synthesis was carried out manually on “RINK” resin, 4-(2′,4′-dimethoxy-phenyl-Fmoc-aminomethyl)-phenoxy resin. Loading was approximately 0.6 mmol/g. Peptide condensation was carried out usingthe steps of: 1) Coupling: two equivalents each of Fmoc-amino acid, HOBt and DCC in DMF for 1-4 hours at room temperature; 2) Recoupling: one equivalent each of Fmoc-amino acid, HOBt and DCC; 3) Acetylation: 20% (v/v) (CH₃CO)₂O/DCM for 1 hour at room temperature; and 4) N-a-Fmoc deprotection: 20% (v/v) piperidine in DMF for 25 minutes.

The removal of side chain protecting groups (tBu, Boc, Trt, Pmc) and cleavage of peptide from the resin were accomplished using a TFA containing cocktail ((v/v) 55/5/40 TFA/anisole/DCM) for 90 minutes at room temperature under N₂. The peptide was precipitated from diethyl ether, filtered and dried. The crude peptide was purified and analyzed by HPLC on a reverse phase column with a gradient elution using 0.06% TFA/H₂O and 0.06% TFA/acetonitrile.

### Example 2

### Hot Plate Assay of Compounds of Formula A

Measurement of Analgesic Activity

For this test, CD #1 male mice weighing between 20 and 25 g were used. The mice were weighed, marked, and divided into groups of 10.

The mice were usually treated by subcutaneous injection of the compound, the standard, or the medium in an injection volume equivalent to 0.1 ml/10 g p.c. (10 ml/kg). If an antagonist such as Nalaxone or N-methyl-Levallorphan was used, it was administered intra-peritoneally 20 minutes before the compound (or the standard, or the medium) was administered. The injection volume was also 0.1 ml/10 g p.c. The dose of the antagonist was 10 mg/kg.

The mice were individually evaluated for reaction time on the hot plate. The temperature of the hot plate (Sorel, model DS37) was set at 55° C. Mice were observed for signs of discomfort such as licking or shaking of the paws, attempting to escape (jumping off the plate) or trembling. The reaction time was counted when one of these signs appeared and was noted in “seconds.” Each mouse was observed for a maximum period of 30 seconds so as to prevent damage to paw tissue. The mice may be observed at different time intervals after administration of the compound, medium or standard, e.g., at 30, 60 or 120 minutes.

For each time reading, the average reaction time of the control group was multiplied by 1.5. The reaction time of each treated mouse was compared to the “control average X 1.5.” If the reaction time was inferior to the “control average X 1.5,” the mouse was considered to not have had an analgesic effect. If the reaction time was superior to the “control average X 1.5” then the mouse was considered to have had an analgesic effect. The number of analgesic mice in a group determined the analgesic percentage of the compound for this reading. If the analgesic percentage waslower than 30%, the compound was considered inactive.

### Example 3

### Writhing Assay of Compounds of Formula A

Measurement of Contortions

The test was performed on CD #1 male mice weighing between 18 and 22 g. Mice were weighed, marked and then injected, by the intraperitoneal route, with 0.3 ml/20 g by weight of a solution of phenylquinone at 0.02%. The contortions which appeared during a 15 minute time period following the injection were counted. The phenylquinone was injected at time intervals of 5, 20 or 60 minutes after administration of the compound (or medium, or standard) by subcutaneous route. It was injected at time intervals of 60 minutes after the administration of the compound (or medium, or standard) by oral route;

The 0.02% phenylquinone solution was prepared as follows: 20 mg of phenylquinone was dissolved in 5 ml ethanol 90% (Sigma). The dissolved phenylquinone was slowly added to 95 ml of distilled water continuously shaken and preheated (not boiled). The phenylquinone solution was, at all times protected from light and a new solution was prepared every day for the test. Each group usually contained 10 mice. If an antagonist, such as naloxone, was used, it was administered 20 minutes before the compound (or the medium, or the standard) by the intraperitoneal route.

TABLE 1

| | | Ki^{μ} | | GPI(IC₅₀) | ED₅₀(PBQ) | Hot Plate |
|---|---|---|---|---|---|---|
| BCH# | Sequence | [nM] | Ki^{δ}Ki^{μ} | [nM] | mg/kg(20 min) | mg/kg |
| 1774 | H-Tyr-D-Ala-Phe-Phe-NH₂ | 1.53 | 409 | 3 | 1.4 | >100 |
| 753 | H-Tyr-D-Phe-Phe-Phe-NH₂ | 3.63 | 37.7 | 247 | >20 | |
| 754 | H-Tyr-Aib-Phe-Phe-NH₂ | | | 73 | >20 | |
| 755 | H-Tyr-D-Nle-Phe-Phe-NH₂ | 0.968 | 373 | 15 | 2.5 (5 min.) | |
| 756 | H-Tyr-Pro-Phe-Phe-NH₂ | 4.10 | 182 | 15 | >20 | |
| 757 | H-Tyr-D-Ala-Phe-2′-Nal-NH₂ | 0.655 | 119 | 2 | 1.1 (5 min.) | |
| 758 | H-Tyr-D-Ala-2′-Nal-1′-Nal-NH₂ | 5.61 | 102 | — | >20 | |
| 1775 | H-Tyr-D-Ala-D-Phe-Phe-NH₂ | 26.0 | 82.7 | 925 | | |
| 1776 | H-Tyr-D-Ala-Phe-Phe(4-NO₂)—NH₂ | 0.509 | 129 | 8 | 4 | |
| 1777 | H-Tyr-D-Ala-Phe(4-NO₂)-Phe(4-NO₂)—NH₂ | 0.826 | 570 | 6 | >20 | |
| 1778 | H-Tyr-D-Ala-Phe-Phe(4-N₃)—NH₂ | 1.49 | 107 | 50 | | |
| 1779 | H-Tyr-D-Ala-Phe(4-NO₂)-Phe-NH₂ | 56.8 | 24.3 | 77 | | |
| 1780 | H-Tyr-D-Ala-Phe-Tic-NH₂ | 12.7 | 279 | — | | |
| 1781 | H-Tyr-D-Ala-Phe-Phe(NMe)-NH₂ | 22.6 | 215 | 241 | | |
| 1782 | H-Tyr-D-Ala-Phe-1′-Nal-NH₂ | 0.981 | 174 | 2 | >20 | |
| 1783 | H-Tyr-D-Ala-1′-Nal-1′-Nal-NH₂ | 2.88 | 410 | — | >20 | |
| 1784 | H-Tyr-D-Ala-Trp-Phe-NH₂ | 3.57 | 238 | 20 | >20 | |
| 1785 | H-Tyr-D-Ala-Phe-Trp-NH₂ | 2.21 | 214 | 16 | >20 | |
| 1786 | H-Tyr-D-Ala-Trp-Trp-NH₂ | 0.833 | 783 | | 10 | |
| 1787 | H-Tyr-∇Ala-Phe-Phe-NH₂ | | | | 10 | |
| 2202 | ∇CH₂Tyr-D-Ala-Phe-Phe-NH₂ | | | | >10 | |
| 2208 | H-Tyr-D-Nle-Phe-Trp-NH₂ | | | | >3 | |
| 2211 | H-Tyr-D-Nle-Phe-2′-Nal-NH₂ | | | | >10 | |
| 2212 | H-Tyr-D-Nle-Trp-Phe-NH₂ | | | | >10 | |
| 2213 | H-Tyr-D-Ala-Trp-2′-Nal-NH₂ | | | | >5 | |
| 2214 | H-Tyr-D-Nle-Trp-2′-Nal-NH₂ | | | | 15 | |
| 2217 | H-Tyr-D-Nle-Trp-Trp-NH₂ | | | | >5 | |
| 2462 | H-Tyr-D-Nva-Phe-Phe-NH₂ | | | | 2.7 | >100 |
| 2463 | H-Tyr-D-Ser-Phe-Phe-NH₂ | 2.2 | | 13 | 0.5 | >100 |
| 2464 | H-Tyr-D-Val-Phe-Phe-NH₂ | | | | >10 | |
| 2465 | H-Tyr-D-Leu-Phe-Phe-NH₂ | | | | >10 | |
| 2473 | H-Tyr-D-Ile-Phe-Phe-NH₂ | | | | >10 | |
| 2577 | H-Tyr-D-Abu-Phe-Phe-NH₂ | | | | >10 | |
| 2578 | H-Tyr-Chl-Phe-Phe-NH₂ | | | | >10 | |
| 2579 | H-Tyr-Cle-Phe-Phe-NH₂ | | | | >10 | |
| 2687 | H-Tyr-D-Arg-Phe-Phe-NH₂ | 0.88 | 2480 | 8.71 | 0.5 | >100 |
| 2690 | H-Tyr-D-Cys-Phe-Phe-NH₂ | | | | 6.2 | >100 |
| 2811 | H-Tyr-D-Thr-Phe-Phe-NH₂ | | | | 12 | |
| 2813 | H-Dmt-D-Ser-Phe-Phe-NH₂ | | | | 0.15 | −40 |
| 3237 | H-Dmt-D-Ala-Phe-Phe-NH₂ | 0.16 | 26.4 | 0.34 | | |
| 3238 | H-Dmt-D-Ala-Phe-Phe-NH₂ | 71.8 | 3.1 | | | |
| 3240 | H-Tyr-D-Ala-Phe-Cys(Bzl)NH₂ | 5.3 | 57.3 | 9.16 | | |
| 3241 | H-Tyr-D-Arg-Phe-Cys(Bzl)NH₂ | 6.95 | 46.8 | 33.05 | | |

### Example 4

### Preparation of H-Tyr-D-Ala-Phe(pF)-Phe-NH

The synthetic peptide was prepared using Knorr resin. The amino acids used had their alpha amino group Fmoc-protected and Tyrosine side chain tBu protected. Dimethylformamide used in the coupling step was free of dimethylamine. DNT used for the washing steps and TFA were Biograde purity. For the purification step USP purified H₂O and acetonitrile of HPLC grade were used. All remaining solvents were of ACS purity and used as such without any purification. Solid phase synthesis was carried out manually on the resin having a load of 0.84 mM/g. Peptide condensation was carried out using 1.5 to 2 equivalents each of Fmoc-amino acid, HOBT and BOP in DMF for 3-24 hours at room temperature. The alpha amino Fmoc deprotection steps were performed using 20% (v/v) Piperidine in DMF for 25 minutes. The peptide cleavage and side chain deprotection were accomplished by treatment with TFA/CH₂Cl₂/anisole. The peptide resin was treated with TFA for two periods of 90 minutes at room temperature under nitrogen atmosphere. After CH₂CL₂ washing and evaporation the residue was treated with ethyl ether, the precipitate filtered and dried under vacuum.

The crude peptide obtained was purified by HPLC on a C_{18 10}μ-15μ 300A reverse phase column, with a gradient elution of 0.06% TFA/H₂O to 0.06% TFA/Acetonitrile. Monitoring was performed at 220 nm. Pure fractions were pooled and lyophilized. The purified material was exchanged into its hydrochloride salt form to give the pure title compound.

In a like manner the following peptides were also synthesized:

1A H-Tyr-D-Ala-Phe-Phe-NH₂

1B H-Tyr-D-Ala-Phe(pF)-Phe(pF)-NH₂

### Example 5

### Preparation of H-Tyr-D-Arg-Phe(pF)-Phe-NH₂

The synthetic peptide was prepared using Knorr resin. The amino acids used had their alpha amino group Fmoc-protected. Side chains were protected using Pmc for D-Arginine, and tBu for Tyrosine. Dimethylormamide used in the coupling step was free of dimethylamine. DMF used for the washing steps and TFA were Biograde purity. For the purification step USP purified H₂O and acetonitrile of HPLC grade were used. All remaining solvents were of ACS purity and used as such without any further purification.

Solid phase synthesis was carried out manually on the resin with a load of 0.84 mM/g. Peptide condensation was carried out using 2 equivalents each of Fmoc-amino acid, HOBT and BOP in DMF for 2-5 hours at room temperature. The alpha amino Fmoc deprotection steps were performed using 20% (v/v) piperidine in DMF for 25 minutes. Peptide cleavage and side chain deprotection were accomplished by TFA/CH₂Cl/anisole treatment. The peptide resin was treated with TFA for two periods of 90 minutes at room temperature under nitrogen atmosphere. After CH₂Cl₂ washing and evaporation the residue was treated with ethyl ether. The precipitate was then filtered and dried under vacuum.

The crude peptide obtained was purified by HPLC on a C₁₈ 10β-15μ 300A reverse phase column, with gradient elution using from 0.06% TFA/H₂O to 0.06% TFA/acetonitrile. Monitoring was performed at 220 nm. Pure fractions were pooled and lyophilized.

In a like manner the following peptide compounds were synthesized:

2A H-Tyr-D-Arg-Phe-Phe-NH₂

2B H-Tyr-D-Arg-Phe(pF)-Phe(pF)-NH₂

### Example 6

### Radioligand Binding Assay of Compounds of Formula B

Membrane Preparation

Male Sprague-Dawley rats weighing between 350-450 g were sacrificed by inhalation of CO₂. The rats were decapitated and the brains (minus the cerebellum) were removed, placed in ice-cold saline solution, and then homogenized in ice-cold 50 mM Tris buffer pH 7.4 (10 ml/brain). Membranes were centrifuged at 14000 rpm for 30 min. at 4° C. The pellets were re-suspended in approximately 6 ml/brain of ice-cold Tris buffer 50 mM pH 7.4 and stored at −78° C. until ready for use. Protein quantification of the brain homogenate was conducted according to protein assay kit purchased (Bio-Rad).

Radioligand Inhibition

(³H)-DAMGO and (³H)-DAGLE were used as radioligands for the μ and δ receptors, respectively. Radioligand 50 μl, membranes 100 μl, and serially diluted test compound were incubated for 1 hr at 22° C. Nonspecific binding was determined using a 500 fold excess of unlabeled ligand in the presence of tracer and membranes. Free ligand was separated from bound by filtration through Whatman GF/B paper (presoaked in polyethylenimine 1% aqueous solution) and rinsing with ice-cold 50 mM Tris pH 7.4 using a Brandel cell harvester. The filters were dried and radioactivity was counted in a 24 well microplate in the presence of 500 μl scintillant per well. Radioactivity was measured using a Wallace 1450 Microbeta counter. Ki's for the various compounds were determined from the IC₅₀ according to the Cheng and Prusoff equation. Results of the binding assay are summarized in Table 2.

The activity of the peptide compounds on μ receptors was determined using the Guinea Pig Ileum (GPI) assay (longitudinal muscle preparation) according to the procedures described in Schiller et al., Biophys. Res. Commun. 85:1322 (1975). Activity results are summarized in Table 2.

### Example 7

### Hot Plate Assay of Compounds of Formula B

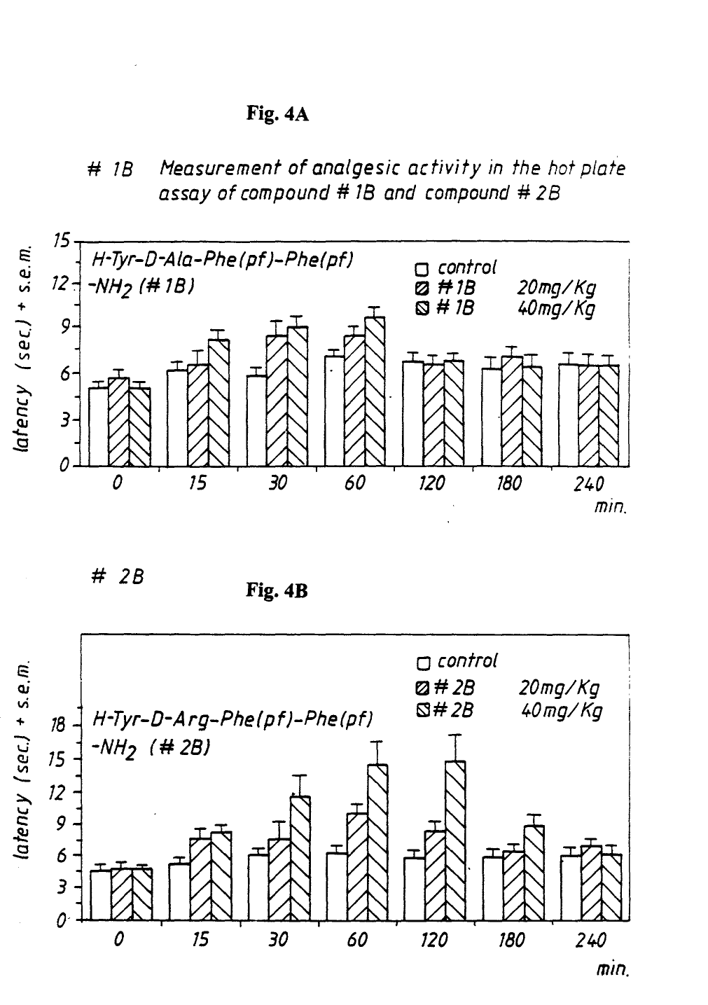

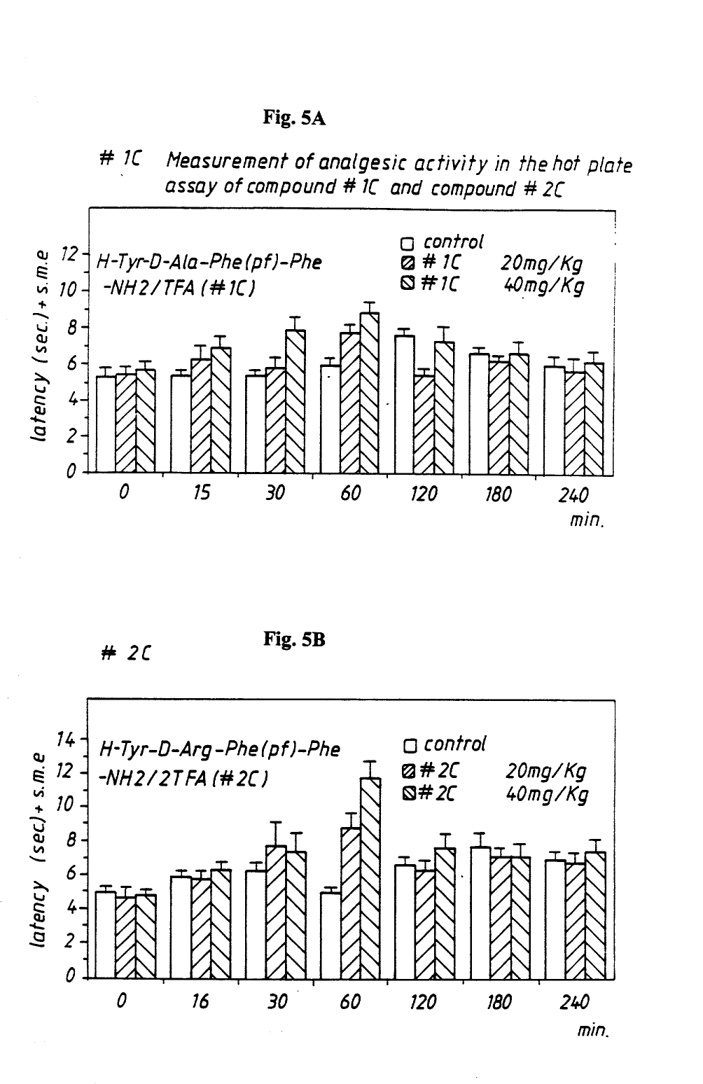

For hot plate assays, CD #1 male mice weighing between 20 and 25 g were used. The mice were weighed, marked, and divided into groups of 10. Mice were treated by subcutaneous injection of the compound (or the standard or the medium) in an injection volume equivalent to 0.1 ml/10 g (10 ml/kg). The remaining conditions of the experiment and calculations were as described in Example 2. Results are shown in FIGS. 4 to 6.

### Example 8

### Writhing Assay of Compounds of Formula B

Writhing assays were performed on CD#1 male mice weighing between 12 and 22 g. Assays were carried out as described in Example 3 and results are summarized below in Table 2. It can be seen that the peptides in which either one or both of R₃ and R₄ are Phe(pF) exhibit greater selectivity for the μ opioid receptor compared to the corresponding. compound without Phe(pF). In addition, these peptides exhibit a greater transduction of the receptor as determined in the GPI assay and greater peripheral analgesic activity as determined in the writhing assay.

TABLE 2

| | Binding Assay | | | Writhing | CPI |
|---|---|---|---|---|---|
| Peptide | Kᵢμ (nM) | Kᵢδ (nM) | Kᵢδ Kᵢμ | ED₅₀ (mg/kg) | IC₅₀ (nM) |
| 1A | 1.53 | 625.8 | 409 | 1.4 | 3 |
| 1B | 0.2 | 199.6 | 998 | 0.2 | 0.12 |
| 1C | 0.36 | 201.2 | 559 | 0.5 | |
| 2A | 0.68 | 1652.6 | 2430 | 0.5 | 6.7 |
| 2B | 0.22 | >1000 | | 0.3 | |
| 2C | 0.57 | 952.5 | 1671 | 0.3 | 1.52 |

### Example 9

### 58° C. Hot Plate Assay of Compounds of Formula B

For this assay, NMR1 male mice weighing between 20 and 25 g were used. The mice were weighed, marked, and divided into groups of 6. They were then treated by subcutaneous injection of the compound (or the standard or the medium) in an injection volume equivalent to 0.1 ml 10 g p.c. (10 ml/kg).

The mice were individually evaluated for reaction time on the hot plate. The temperature of the hot plate (IITC, Inc; Model 35-0) was set at 58° C. The mouse was observed for signs of discomfort such as licking or shaking of the paws, attempting to escape (jumping off the plate) or trembling. The reaction time was counted when one of these signs appeared and was noted in “seconds”. Each mouse was observed for a maximum period of 20 seconds so as to prevent damage to the paw tissue.

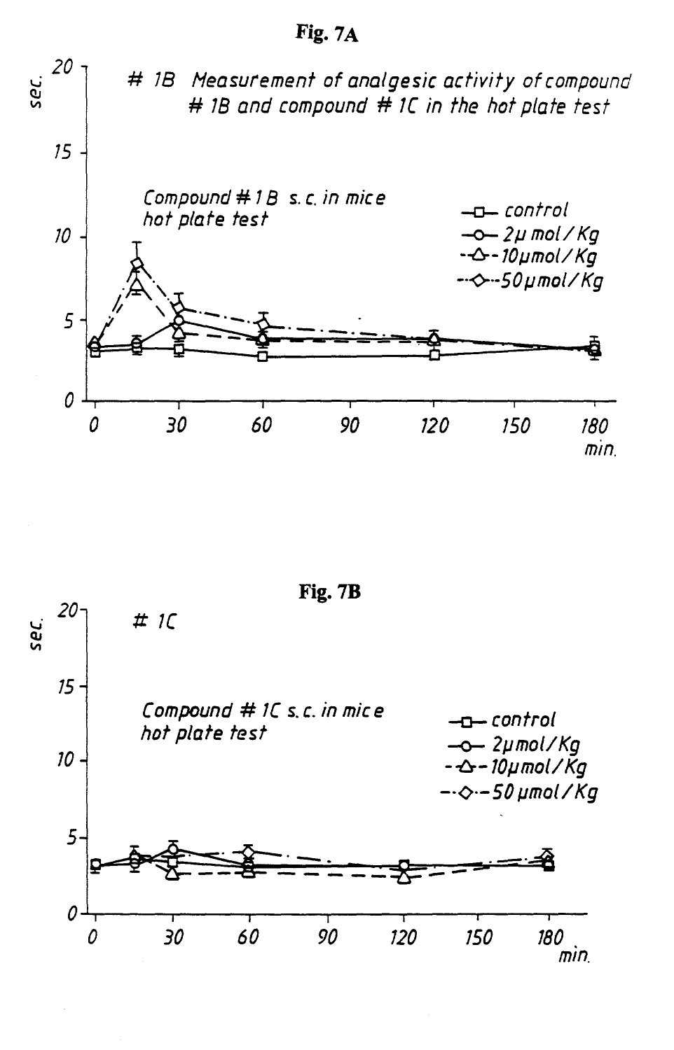

The compound was considered analgesic if the reaction time was significantly different (p<0.05; two way ANOVA, sigma slot) from the control group. Results are shown in FIG. 7.

### Example 10

### Tail Flick Assay of Compounds of Formula B

For this assay, NMRI male mice weighing between 20 and 25 g were used. The mice were weighed, marked, and divided into group of 6. The mice were treated by subcutaneous injection of the compounds (or the standard medium) in an injection volume equivalent to 0.1 ml/10 g p.c (10 ml/kg).

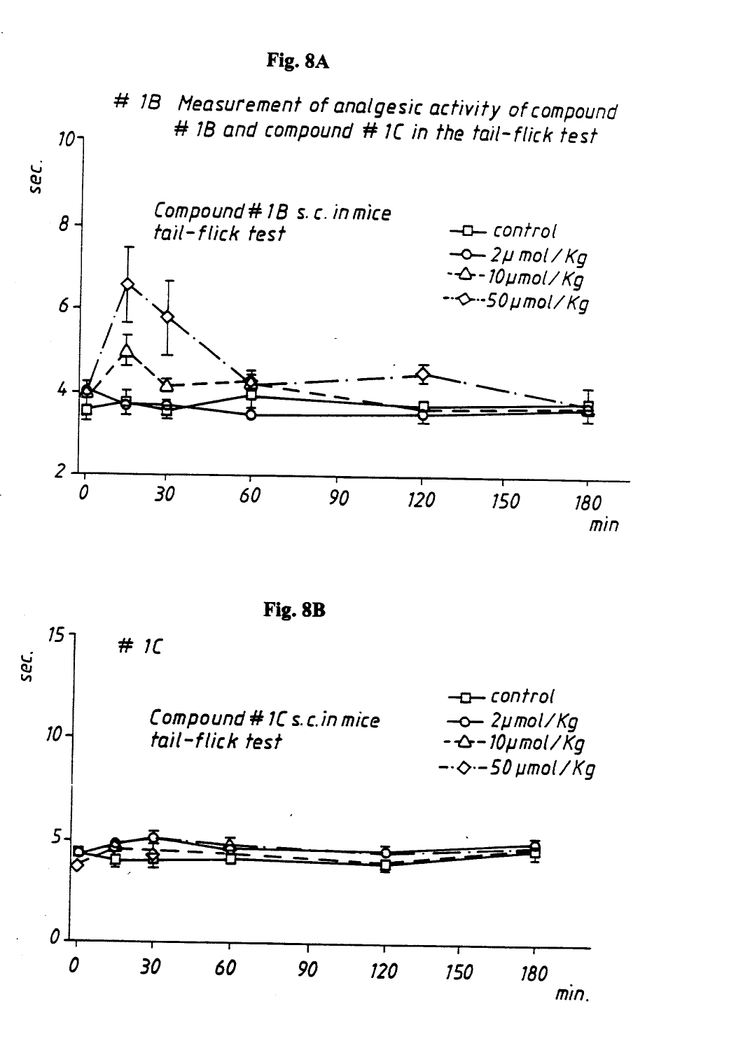

Mice were individually evaluated for reaction time in the tail flick test. The latency to the flick of the tail was measured when a rheostat-controlled light beam was directed at the tip of the tail (HTC Inc. Model 33). Each mouse was observed for a maximum period of 10 seconds so as to prevent damage to the tissue. Results are shown in FIG. 8.

What is claimed is:

1. A compound of having the chemical structure of formula (A): 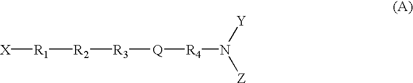
with peripheral analgesic effect, wherein:
a) X is selected from the group consisting of H and C₁₋₆ alkyl;
b) Y and Z are independently selected from the group consisting of H, cyclic aralkyl, and C₁₋₆ alkyl;
C) R₁ is a tyrosyl residue or a 2′,6′-dimethyltyrosyl residue;
d) R₂ is an amino acid having the R-configuration, aminoisobutyric acid, cyclopropylalanine, cyclohomoleucine or cycloleucine;
e) R₃ is an aromatic amino acid;
f) R₄ is an aromatic amino acid residue;
g) Q is an amide bond or an interposed amide bond mimetic;
h) with the proviso that when:
i) R1 is a tyrosyl residue;
ii) R₂ is D-alanine;
iii) X, Y, and Z are H; and
iv) R₃ is phenylalanine;
then R₄ is not unsubstituted phenylalanine or phenylalanine substituted with 4NO₂ or 4N₃;
i) with the further proviso that when:
i) R₁ is a tyrosyl residue;
ii) R₂ is D-alanine;
iii) X, Y, and Z are H; and
iv) R₄ is phenylalanine;
then R₃ is not unsubstituted phenylalanine or phenylalanine substituted with 4NO₂;
j) with the further proviso that when:
i) R₁ is a tyrosyl residue;
ii) R₂ is D-alanine;
iii) X, Y, and Z are H; and
iv) R₄ is 1′-naphthylalanine;
then R₃ is not 1′-naphthylalanine or 2′-naphthylalanine;
k) with the further proviso that when:
i) R₁ is a tyrosyl residue;
ii) R₂ is D-alanine; and
iii) X, Y and Z are H,
then both R₃ and R₄ are not tryptophan;
l) with the further proviso that when:
i) R₁ is a tyrosyl residue;
ii) R₂ is a D-amino acid with a lower alkyl or lower thioalkyl group as a side chain; and
iii) R₄ is a neutral amino acid,
then R₃ is not unsubstituted phenylalanine;
m) and wherein said compound is not selected from the group consisting of:
H-Tyr-D-Phe-Phe-Phe-NH₂;
H-Tyr-D-NMePhe-Phe-Phe-NH₂;
H-Tyr-D-Tic-Phe-Phe-NH₂;
H-Tyr-Pro-Phe-Thr(Bzl)-NH₂;
H-Tyr-Pro-Phe-Phe-NH₂;
H-Tyr-Pro-Phe-Apb-NH₂;
H-Tyr-Pro-Phe-App-NH₂;
H-Tyr-Pro-Phe-Aph-NH₂; and
H-Tyr-Pro-Apb-Phe-NH₂;
wherein Apb is 2-amino4-phenylbutanoic acid, App is 2-amino-5-phenylpentanoic acid and Aph is 2-amino-6-phenylhexanoic acid.

2. The compound of claim 1, wherein X is H.

3. The compound of either claim 1 or 2, wherein:
a) R₂ is as defined in claim 1;
b) with the proviso that when:
i) R₁ is a tyrosyl residue;
ii) R₂ is D-alanine; and
iii) Y and Z are H;
then R₃ and R₄ are different and are selected from the group consisting of phenylalanine, and tryptophan.

4. The compound of either claim 1 or claim 2, wherein Q is an amide bond or an interposed amide bond mimetic of the formula Q₁-Q₂, wherein:
a) Q₁ is selected from the group consisting of CH₂, CHOH, C═O, C═S, and CH═; and
b) Q₂ is selected from the group consisting of CH₂, NH, S, SO, SO₂, O, and CH═;
c) with the proviso that when Q₁ is CH═, then Q₂ is CH═.

5. The compound of claim 3, wherein Q is an amide bond or an interposed amide bond mimetic of the formula Q₁-Q₂, wherein:
a) Q₁ is selected from the group consisting of CH₂, CHOH, C═O, C═S, and CH═; and
b) Q₂ is selected from the group consisting of CH₂, NH, S, SO, SO₂, O, and CH═;
c) with the proviso that when Q₁ is CH═, then Q₂ is CH═.

6. The compound of claim 5, wherein:
a) Y and Z are H;
b) R₂ is as defined in claim 1;
c) R₃ is an aromatic amino acid; and
d) R₄ is an aromatic amino acid;
e) with the proviso that when:
i) R₁ is a tyrosyl residue; and
ii) R₂ is D-alanine;
then R₃ and R₄ are different and are selected from the group consisting of phenylalanine and tryptophan.

7. The compound of either claim 1 or claim 2, wherein:
a) Y and Z are H;
b) R₂ is as defined in claim 1;
c) R₃ is an aromatic amino acid; and
d) R₄ is an aromatic amino acid;
e) with the proviso that when:
i) R₁ is a tyrosyl residue; and
ii) R₂ is D-alanine;
then R₃ and R₄ are different and are selected from the group consisting of phenylalanine and tryptophan.

8. The compound of claim 4, wherein:
a) Y and Z are H;
b) R₂ is as defined in claim 1;
c) R₃ is an aromatic amino acid; and
d) R₄ is an aromatic amino acid;
e) with the proviso that when:
i) R, is a tyrosyl residue; and
ii) R₂ is D-alanine;
then R₃ and R₄ are different and are selected from the group consisting of phenylalanine and tryptophan.

9. The compound of claim 6, wherein:
a) R₂ is as defined in claim 1, with the proviso that R₂ is not D-alanine;
b) R₃ is a phenylalanyl residue; and
c) R₄ is a phenylalanyl residue.

10. The compound of claim 7, wherein:
a) R₂ is as defined in claim 1, with the proviso that R₂ is not D-alanine;
b) R₃ is a phenylalanyl residue; and
c) R₄ is a phenylalanyl residue.

11. The compound of claim 8, wherein:
a) R₂ is as defined in claim 1, with the proviso that R₂ is not D-alanine;
b) R₃ is a phenylalanyl residue; and
c) R₄ is a phenylalanyl residue.

12. The compound of claim 6, wherein:
a) R₁ is a tyrosyl residue;
b) R₂ is selected from the group consisting of D-serine and D-arginine;
c) R₃ is a phenylalanyl residue;
d) R₄ is a phenylalanyl residue; and
e) Q is an amide bond.

13. The compound of claim 7, wherein:
a) R₁ is a tyrosyl residue;
b) R₂ is selected from the group consisting of D-serine and D-arginine;
c) R₃ is a phenylalanyl residue;
d) R₄ is a phenylalanyl residue; and
e) Q is an amide bond.

14. The compound of claim 8, wherein:
a) R₁ is a tyrosyl residue;
b) R₂ is selected from the group consisting of D-serine and D-arginine;
c) R₃ is a phenylalanyl residue;
d) R₄ is a phenylalanyl residue; and
e) Q is an amide bond.

15. A compound selected from the group consisting of:
H-Tyr-Aib-Phe-Phe-NH₂;
H-Tyr-D-Nle-Phe-Phe-NH₂;
H-Tyr-D-Ala-Phe-2′-Nal-NH₂;
H-Tyr-D-Ala-D-Phe-Phe-NH₂;
H-Tyr-D-Ala-Phe(4NO₂)-Phe(4NO₂)—NH₂;
H-Tyr-D-Ala-Phe-Tic-NH₂;
H-Tyr-D-Ala-Phe-Phe(NMe)-NH₂;
H-Tyr-D-Ala-Phe-1′-Nal-NH₂;
H-Tyr-D-Ala-Trp-Phe-NH₂;
H-Tyr-D-Ala-Phe-Trp-NH₂;
H-Tyr-VAla-Phe-Phe-NH₂;
VCH₂-Tyr-D-Ala-Phe-Phe-NH₂;
H-Tyr-D-Nle-Phe-Trp-NH₂;
H-Tyr-D-Nle-Phe-2′-Nal-NH₂;
H-Tyr-D-Nle-Trp-Phe-NH₂;
H-Tyr-D-Ala-Trp-2′-Nal-NH₂;
H-Tyr-D-Nle-Trp-2′-Nal-NH₂;
H-Tyr-D-Nle-Trp-Trp-NH₂;
H-Tyr-D-Nva-Phe-Phe-NH₂;
H-Tyr-D-Ser-Phe-Phe-NH₂;
H-Tyr-D-Val-Phe-Phe-NH₂;
H-Tyr-D-Leu-Phe-Phe-NH₂;
H-Tyr-D-Ile-Phe-Phe-NH₂;
H-Tyr-D-Abu-Phe-Phe-NH₂;
H-Tyr-Chl-Phe-Phe-NH₂;
H-Tyr-Cle-Phe-Phe-NH₂;
H-Tyr-D-Arg-Phe-Phe-NH₂;
H-Tyr-D-Cys-Phe-Phe-NH₂;
H-Tyr-D-Thr-Phe-Phe-NH₂;
H-DMT-D-Ser-Phe-Phe-NH₂;
H-Tyr-D-Ala-Phe-Phe-OH trifluoroacetate;
H-Tyr-D-Ala-Phe-Phg-NH₂ trifluoroacetic acid salt;
H-Tyr-D-Arg-Phe-Hph-NH₂ bis-trifluoroacetic acid;
H-DMT-D-Ala-Phe-Phe-NH₂ trifluoroacetic acid;
H-D-DMT-D-Ala-Phe-Phe-NH₂ trifluoroacetic acid salt;
H-Tyr-D-Ala-Phe-Hph-NH₂ trifluoroacetic acid salt;
H-Tyr-D-Ala-Phe-Cys(Bzl)-NH₂ trifluoroacetic acid salt;
H-Tyr-D-Arg-Hph-Phe-NH₂ bis-trifluoroacetic acid salt;
H-Tyr-D-Arg-Phg-Phe-NH₂ bis-trifluoro acetic acid salt;
H-Tyr-D-Ala-Phe-Phe-CH₂OH hydrochloride salt;
H-Tyr-D-Ala-Hph-Phe-NH₂ trifluoroacetic acid salt;
H-Tyr-D-Met-Phe-Phe-NH₂ trifluoroacetic acid salt;
H-Tyr-D-Arg-Phe-D-Phe-NH₂ bis-trifluoroacetic acid salt;
H-Tyr-D-Ala-Phg-Phe-NH₂ trifluoroacetic acid salt;
H-Tyr-(D)-Ala-(D)-Phg-Phe-NH₂ trifluoroacetic acid salt;
H-Tyr-D-Arg-Phe-Phe-(D)-NH₂ bis-trifluoroacetic acid salt;
H-Tyr-D-Arg-Phe-D-Phe(pF)-NH₂ ditrifluoroacetic acid salt;
H-Tyr-D-Ala-Phe-Phe(pF)-NH₂ trifluoroacetic acid salt; and
H-Tyr-D-Ala-Phe-D-Phe(pF)-NH₂ trifluoroacetic acid salt.

16. The compound of claim 1, wherein said compound is H-Tyr-D-Ser-Phe-Phe-NH₂.

17. The compound of claim 1, wherein said compound is H-Tyr-D-Arg-Phe-Phe-NH₂.

18. A pharmaceutical composition possessing analgesic activity, comprising, in admixture with a pharmaceutically acceptable carrier, an effective amount of at least one compound having the chemical structure of formula (A): 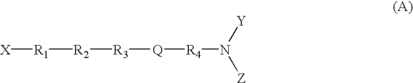
with peripheral analgesic effect, wherein:
a) X is-selected from the group consisting of H and C₁₋₆ alkyl;
b) Y and Z are independently selected from the group consisting of H, cyclic aralkyl, and C₁₋₆ alkyl;
c) R₂ is a tyrosyl residue or a 2′,6′-dimethyltyrosyl residue;
d) R₂ is an amino acid having the R-configuration, aminoisobutyric acid, cyclopropylalanine, cyclohomoleucine or cycloleucine;
e) R₃ is an aromatic amino acid;
f) R₄ is an aromatic amino acid residue;
g) Q is an amide bond or an interposed amide bond mimetic;
h) with the proviso that when:
i) R₁ is a tyrosyl residue;
ii) R₂ is D-alanine;
iii) X, Y, and Z are H; and
iv) R₃ is phenylalanine;
then R₄ is not unsubstituted phenylalanine or phenylalanine substituted with 4NO₂ or 4N₃;
i) with the further proviso that when:
i) R₁ is a tyrosyl residue;
ii) R₂ is D-alanine;
iii) X, Y, and Z are H; and
iv) R₄ is phenylalanine;
then R₃ is not unsubstituted phenylalanine or phenylalanine substituted with 4NO₂;
j) with the further proviso that when:
i) R₁ is a tyrosyl residue;
ii) R₂ is D-alanine;
iii) X, Y, and Z are H; and
iv) R₄ is 1′-naphthylalanine;
then R₃ is not 1′-naphthylalanine or 2′-naphthylalanine;
k) with the further proviso that when:
i) R₁ is a tyrosyl residue;
ii) R₂ is D-alanine; and
iii) X, Y and Z are H,
then both R₃ and R₄ are not tryptophan;
l) with the further proviso that when:
i) R₁ is a tyrosyl residue;
ii) R₂ is a D-amino acid with a lower alkyl or lower thioalkyl group as a side chain; and
iii) R₄ is a neutral amino acid,
then R₃ is not unsubstituted phenylalanine;
m) and wherein said compound is not selected from the group consisting of:
H-Tyr-D-Phe-Phe-Phe-NH₂;
H-Tyr-D-NMePhe-Phe-Phe-NH₂;
H-Tyr-D-Tic-Phe-Phe-NH₂;
H-Tyr-Pro-Phe-Thr(Bzl)-NH₂;
H-Tyr-Pro-Phe-Phe-NH₂;
H-Tyr-Pro-Phe-Apb-NH₂;
H-Tyr-Pro-Phe-App-NH₂;
H-Tyr-Pro-Phe-Aph-NH₂; and
H-Tyr-Pro-Apb-Phe-NH₂;
wherein Apb is 2-amino4-phenylbutanoic acid, App is 2-amino-5-phenylpentanoic acid and Aph is 2-amino-6-phenylhexanoic acid.

19. The pharmaceutical composition of claim 18 wherein said composition has peripheral analgesic activity and wherein said compound has a chemical structure in which X is H.

20. The pharmaceutical composition of either claim 18 or claim 19 wherein said composition has peripheral analgesic activity and wherein said compound has a chemical structure in which:
a) R₂ is as defined in claim 18;
b) with the proviso that when:
i) R₁ is a tyrosyl residue;
ii) R₂ is D-alanine; and
iii) Y and Z are H;
then R₃ and R₄ are different and are selected from the group consisting of phenylalanine, and tryptophan.

21. The pharmaceutical composition of either claim 18 or claim 19 wherein said composition has peripheral analgesic activity and wherein said compound has a chemical structure in which: Q is an amide bond or an interposed amide bond mimetic of the formula Q₁—Q₂, wherein:
a) Q₁ is selected from the group consisting of CH₂, CHOH, C═O, C—S, and CH═; and
b) Q₂ is selected from the group consisting of CH₂, NH, S, SO, SO₂, O, and CH═;
c) with the proviso that when Q₁ is CH═, then Q₂ is CH═.

22. The pharmaceutical composition of 20 wherein said composition has peripheral analgesic activity and wherein said compound has a chemical structure in which: Q is an amide bond or an interposed amide bond mimetic of the formula Q₁-Q₂, wherein:
a) Q₁ is selected from the group consisting of CH₂, CHOH, C═O, C═S, and CH═; and
b) Q₂ is selected from the group consisting of CH₂, NH, S, SO, SO₂, O, and CH═;
c) with the proviso that when Q₁ is CH═, then Q₂ is CH═.

23. The pharmaceutical composition of claim 22, wherein said composition has peripheral analgesic activity and wherein said compound has a chemical structure in which:
a) Y and Z are H;
b) R₂ is as defined in claim 18;
c) R₃ is an aromatic amino acid; and
d) R₄ is an aromatic amino acid;
e) with the proviso that when:
i) R₁ is a tyrosyl residue; and
ii) R₂ is D-alanine;
then R₃ and R₄ are different and are selected from the group consisting of phenylalanine and tryptophan.

24. The pharmaceutical composition of either claim 18 or 19, wherein said composition has peripheral analgesic activity and wherein said compound has a chemical structure in which:
a) Y and Z are H;
b) R₂ is as defined in claim 18;
c) R₃ is an aromatic amino acid; and
d) R₄ is an aromatic amino acid;
e) with the proviso that when:
i) R₁ is a tyrosyl residue; and
ii) R₂ is D-alanine;
then R₃ and R₄ are different and are selected from the group consisting of phenylalanine and tryptophan.

25. The pharmaceutical composition of claim 18, further comprising an effective amount of at least one other therapeutically active agent.

26. The pharmaceutical composition of claim 20, further comprising an effective amount of at least one other therapeutically active agent.

27. The pharmaceutical composition of claim 21, further comprising an effective amount of at least one other therapeutically active agent.

28. The pharmaceutical composition of claim 23, further comprising an effective amount of at least one other therapeutically active agent.

29. The pharmaceutical composition of claim 24, further comprising an effective amount of at least one other therapeutically active agent.

30. A method for the treatment of pain comprising the step of administering to a mammal in need of such treatment a pharmaceutically effective amount of at least one compound having the chemical structure of formula (A): 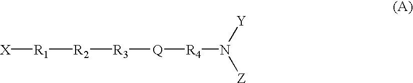
wherein:
a) X is selected from the group consisting of H and C₁₋₆ alkyl;
b) Y and Z are independently selected from the group consisting of H, cyclic aralkyl, and C₁₋₆ alkyl;
c) R₁ is a tyrosyl residue or a 2′,6′-dimethyltyrosyl residue;
d) R₂ is an amino acid having the R-configuration, aminoisobutyric acid, cyclopropylalanine, cyclohomoleucine or cycloleucine;
e) R₃ is an aromatic amino acid;
f) R₄ is an aromatic amino acid residue;
g) Q is an amide bond or an interposed amide bond mimetic;
h) with the proviso that when:
i) R₁ is a tyrosyl residue;
ii) R₂ is D-alanine;
iii) X, Y, and Z are H; and
iv) R₃ is phenylalanine;
then R₄ is not unsubstituted phenylalanine or phenylalanine substituted with 4NO₂ or 4N₃;
i) with the further proviso that when:
i) R₁ is a tyrosyl residue;
ii) R₂ is D-alanine;
iii) X, Y, and Z are H; and
iv) R₄ is phenylalanine;
then R₃ is not unsubstituted phenylalanine or phenylalanine substituted with 4NO₂;
j) with the further proviso that when:
i) R1 is a tyrosyl residue;
ii) R₂ is D-alanine;
iii) X, Y, and Z are H; and
iv) R₄ is 1′-naphthylalanine;
then R₃ is not 1′-naphthylalanine or 2′-naphthylalanine;
k) with the further proviso that when:
i) R₁ is a tyrosyl residue;
ii) R₂ is D-alanine; and
iii) X, Y and Z are H,
then both R₃ and R₄ are not tryptophan;
l) with the further proviso that when:
i) R₁ is a tyrosyl residue;
ii) R₂ is a D-amino acid with a lower alkyl or lower thioalkyl group as a side chain; and
iii) R₄ is a neutral amino acid, ·then R₃ is not unsubstituted phenylalanine;
m) and wherein said compound is not selected from the group consisting of:
H-Tyr-D-Phe-Phe-Phe-NH₂;
H-Tyr-D-NMePhe-Phe-Phe-NH₂;
H-Tyr-D-Tic-Phe-Phe-NH₂;
H-Tyr-Pro-Phe-Thr(Bzl)-NH₂;
H-Tyr-Pro-Phe-Phe-NH₂;
H-Tyr-Pro-Phe-Apb-NH₂;
H-Tyr-Pro-Phe-App-NH₂;
H-Tyr-Pro-Phe-Aph-NH₂; and
H-Tyr-Pro-Apb-Phe-NH₂;
wherein Apb is 2-amino-4-phenylbutanoic acid, App is 2-amino-5-phenylpentanoic acid and Aph is 2-amino-6-phenylhexanoic acid.

31. The method of claim 30, wherein said compound has a chemical structure in which X is H.

32. The method of either claim 30 or claim 31, wherein said compound has a chemical structure in which:
a) R₂ is as defined in claim 30;
b) with the proviso that when:
i) R₁ is a tyrosyl residue;
ii) R₂ is D-alanine; and
iii) Y and Z are H;
then R₃ and R₄ are different and are selected from the group consisting of phenylalanine, and tryptophan.

33. The method of either claim 30 or claim 31, wherein said compound has a chemical structure in which: Q is an amide bond or an interposed amide bond mimetic of the formula Q₁—Q₂, wherein:
a) Q₁ is selected from the group consisting of CH₂, CHOH, C═O, C═S, and CH═; and
b) Q₂ is selected from the group consisting of CH₂, NH, S, SO, SO₂, O, and CH═;
c) with the proviso that when Q₁ is CH═, then Q₂ is CH═.

34. The method of claim 32, wherein said compound has a chemical structure in which: Q is an amide bond or an interposed amide bond mimetic of the formula Q₁-Q₂, wherein:
a) Q₁ is selected from the group consisting of CH₂, CHOH, C═O, C═S, and CH═; and
b) Q₂ is selected from the group consisting of CH₂, NH, S, SO, SO₂, O, and CH═;
c) with the proviso that when Q₁ is CH═, then Q₂ is CH═.

35. The method of 34, wherein said compound has a chemical structure in which:
a) Y and Z are H;
b) R₂ is as defined in claim 30;
c) R₃ is an aromatic amino acid; and
d) R₄ is an aromatic amino acid;
e) with the proviso that when:
i) R₁ is a tyrosyl residue; and
ii) R₂ is D-alanine;
then R₃ and R₄ are different and are selected from the group consisting of phenylalanine and tryptophan.

36. The method of either claim 30 or 31, wherein said compound has a chemical structure in which:
a) Y and Z are H;
b) R₂ is as defined in claim 30;
c) R₃ is an aromatic amino acid; and
d) R₄ is an aromatic amino acid;
e) with the proviso that when:
i) R₁ is a tyrosyl residue; and
ii) R₂ is D-alanine;
then R₃ and R₄ are different and are selected from the group consisting of phenylalanine and tryptophan.

37. A method for the treatment of pain comprising the step of administering to a mammal in need of such treatment a pharmaceutically effective amount of a pharmaceutical composition possessing analgesic activity, wherein said pharmaceutical composition comprises, in admixture with a pharmaceutically acceptable carrier, an effective amount of at least one compound having the chemical structure of formula (A): 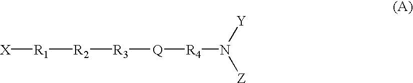
wherein:
a) X is selected from the group consisting of H and C₁₋₆ alkyl;
b) Y and Z are independently selected from the group consisting of H, cyclic aralkyl, and C₁₋₆ alkyl;
c) R₁ is a tyrosyl residue or a 2′,6′-dimethyltyrosyl residue;
d) R₂ is an amino acid having the R-configuration, aminoisobutyric acid, cyclopropylalanine, cyclohomoleucine or cycloleucine;
e) R₃ is an aromatic amino acid;
f) R₄ is an aromatic amino acid residue;
g) Q is an amide bond or an interposed amide bond mimetic;
h) with the proviso that when:
i) R₁ is a tyrosyl residue;
ii) R₂ is D-alanine;
iii) X, Y, and Z are H; and
iv) R₃ is phenylalanine;
then R₄ is not unsubstituted phenylalanine or phenylalanine substituted with 4NO₂ or 4N₃;
i) with the further proviso that when:
i) R₁ is a tyrosyl residue;
ii) R₂ is D-alanine;
iii) X, Y, and Z are H; and
iv) R₄ is phenylalanine;
then R₃ is not unsubstituted phenylalanine or phenylalanine substituted with 4NO₂;
j) with the further proviso that when:
i) R₁ is a tyrosyl residue;
ii) R₂ is D-alanine;
iii) X, Y, and Z are H; and
iv) R₄ is 1′-naphthylalanine;
then R₃ is not 1′-naphthylalanine or 2′-naphthylalanine;
k) with the further proviso that when:
i) R1 is a tyrosyl residue;
ii) R₂ is D-alanine; and
iii) X, Y and Z are H,
then both R₃ and R₄ are not tryptophan;
l) with the further proviso that when:
i) R₁ is a tyrosyl residue;
ii) R₂ is a D-amino acid with a lower alkyl or lower thioalkyl group as a side chain; and
iii) R₄ is a neutral amino acid,
then R₃ is not unsubstituted phenylalanine;
m) and wherein said compound is not selected from the group consisting of:
H-Tyr-D-Phe-Phe-Phe-NH₂;
H-Tyr-D-NMePhe-Phe-Phe-NH₂;
H-Tyr-D-Tic-Phe-Phe-NH₂;
H-Tyr-Pro-Phe-Thr(Bzl)-NH₂;
H-Tyr-Pro-Phe-Phe-NH₂;
H-Tyr-Pro-Phe-Apb-NH₂;
H-Tyr-Pro-Phe-App-NH₂;
H-Tyr-Pro-Phe-Aph-NH₂; and
H-Tyr-Pro-Apb-Phe-NH₂;
wherein Apb is 2-amino-4-phenylbutanoic acid, App is 2-amino-5-phenylpentanoic acid and Aph is 2-amino-6-phenylhexanoic acid.

38. The method of claim 37, wherein said pharmaceutical composition has peripheral analgesic activity and wherein said compound has a chemical structure in which X is H.

39. The method of either claim 37 or claim 38, wherein said pharmaceutical composition has peripheral analgesic activity and wherein said compound has a chemical structure in which:
a) R₂ is as defined in claim 37;
b) with the proviso that when:
i) R₁ is a tyrosyl residue;
ii) R₂ is D-alanine; and
iii) Y and Z are H;
then R₃ and R₄ are different and are selected from the group consisting of phenylalanine, and tryptophan.

40. The method of either claim 37 or claim 38, wherein said pharmaceutical composition has peripheral analgesic activity and wherein said compound has a chemical structure in which:
Q is an amide bond or an interposed amide bond mimetic of the formula Q₁—Q₂, wherein:
a) Q₁ is selected from the group consisting of CH₂, CHOH, C═O, C═S, and CH═; and
b) Q₂ is selected from the group consisting of CH₂, NH, S, SO, S₂O, O, and CH═;
c) with the proviso that when Q₁ is CH═, then Q₂ is CH═.

41. The method of claim 39, wherein said pharmaceutical composition has peripheral analgesic activity and wherein said compound has a chemical structure in which: Q is an amide bond or an interposed amide bond mimetic of the formula Q₁-Q₂, wherein:
a) Q₁ is selected from the group consisting of CH₂, CHOH, C═O, C═S, and CH═; and
b) Q₂ is selected from the group consisting of CH₂, NH, S, SO, SO₂, O, and CH═;
c) with the proviso that when Q₁ is CH═, then Q₂ is CH═.

42. The method of claim 41, wherein said pharmaceutical composition has peripheral analgesic activity and wherein said compound has a chemical structure in which:
a) Y and Z are H;
b) R₂ is as defined in claim 37;
c) R₃ is an aromatic amino acid; and
d) R₄ is an aromatic amino acid;
e) with the proviso that when:
i) R₁ is a tyrosyl residue; and
ii) R₂ is D-alanine;
then-R₃ and R₄ are different and are selected from the group consisting of phenylalanine and tryptophan.

43. The method of either claim 37 or claim 38, wherein said pharmaceutical composition has peripheral analgesic activity and wherein said compound has a chemical structure in which:
a) Y and Z are H;
b) R₂ is as defined in claim 37;
c) R₃ is an aromatic amino acid; and
d) R₄ is an aromatic amino acid;
e) with the proviso that when:
i) R₁ is a tyrosyl residue; and
ii) R₂ is D-alanine;
then R₃ and R₄ are different and are selected from the group consisting of phenylalanine and tryptophan.

44. The method of claim 37, wherein said pharmaceutical composition further comprises an effective amount of at least one other therapeutically active agent.

45. The method of claim 39, wherein said pharmaceutical composition further comprises an effective amount of at least one other therapeutically active agent.

46. The method of claim 40, wherein said pharmaceutical composition further comprises an effective amount of at least one other therapeutically active agent.

47. The method of claim 42, wherein said pharmaceutical composition further comprises an effective amount of at least one other therapeutically active agent.

48. The method of claim 43, wherein said pharmaceutical composition further comprises an effective amount of at least one other therapeutically active agent.

49. A method for the treatment of pain comprising the step of administering to a mammal in need of such treatment, a pharmaceutically effective amount of the compound H-Tyr-D-Ala-Phe-Phe-NH₂ or analogues or pharmaceutically acceptable derivatives thereof.

50. The method of claim 49, wherein said analogue is selected from the group consisting of:
H-Tyr-D-Ala-Phe-Phe(4-NO₂)-NH₂ and H-Tyr-D-Ala-Phe-Phe(4-NO₃)-NH₂.

51. A pharmaceutical composition having analgesic activity, comprising in admixture with a pharmaceutically acceptable carrier, an effective amount of at least one peptide selected from the group consisting of:
H-Tyr-Aib-Phe-Phe-NH₂;
H-Tyr-D-Nle-Phe-Phe-NH₂;
H-Tyr-D-Ala-Phe-2′-Nal-NH₂;
H-Tyr-D-Ala-D-Phe-Phe-NH₂;
H-Tyr-D-Ala-Phe(4NO₂)-Phe(4NO₂)-NH₂;
H-Tyr-D-Ala-Phe-Tic-NH₂;
H-Tyr-D-Ala-Phe-Phe(NMe)-NH₂;
H-Tyr-D-Ala-Phe-1′Nal-NH₂;
H-Tyr-D-Ala-Trp-Phe-NH₂;
H-Tyr-D-Ala-Phe-Trp-NH₂;
H-Tyr-VAla-Phe-Phe-NH₂;
VCH₂-Tyr-D-Ala-Phe-Phe-NH₂;
H-Tyr-D-Nle-Phe-Trp-NH₂;
H-Tyr-D-Nle-Phe-2′-Nal-NH2;
H-Tyr-D-Nle-Trp-Phe-NH₂;
H-Tyr-D-Ala-Trp-2′-Nal-NH₂;
H-Tyr-D-Nle-Trp-2′-Nal-NH₂;
H-Tyr-D-Nle-Trp-Trp-NH₂;
H-Tyr-D-Nva-Phe-Phe-NH₂;
H-Tyr-D-Ser-Phe-Phe-NH₂;
H-Tyr-D-Val-Phe-Phe-NH₂;
H-Tyr-D-Leu-Phe-Phe-NH₂;
H-Tyr-D-Ile-Phe-Phe-NH₂;
H-Tyr-D-Abu-Phe-Phe-NH₂;
H-Tyr-Chl-Phe-Phe-NH₂;
H-Tyr-Cle-Phe-Phe-NH₂;
H-Tyr-D-Arg-Phe-Phe-NH₂;
H-Tyr-D-Cys-Phe-Phe-NH₂;
H-Tyr-D-Thr-Phe-Phe-NH₂;
H-DMT-D-Ser-Phe-Phe-NH₂;
H-Tyr-D-Ala-Phe-Phe-OH trifluoroacetate;
H-Tyr-D-Ala-Phe-Phg-NH₂ trifluoroacetic acid salt;
H-Tyr-D-Arg-Phe-Hph-NH₂ bis-trifluoroacetic acid;
H-DMT-D-Ala-Phe-Phe-NH₂trifluoroacetic acid;
H-D-DMT-D-Ala-Phe-Phe-NH₂ trifluoroacetic acid salt;
H-Tyr-D-Ala-Phe-Hph-NH₂trifluoroacetic acid salt;
H-Tyr-D-Ala-Phe-Cys(Bzl)-NH₂ trifluoroacetic acid salt;
H-Tyr-D-Arg-Hph-Phe-NH₂ bis-trifluoroacetic acid salt;
H-Tyr-D-Arg-Phg-Phe-NH₂ bis-trifluoro acetic acid salt;
H-Tyr-D-Ala-Phe-Phe-CH₂OH hydrochloride salt;
H-Tyr-D-Ala-Hph-Phe-NH₂ trifluoroacetic acid salt;
H-Tyr-D-Met-Phe-Phe-NH₂ trifluoroacetic acid salt;
H-Tyr-D-Arg-Phe-D-Phe-NH₂bis-trifluoroacetic acid salt;
H-Tyr-D-Ala-Phg-Phe-NH₂ trifluoroacetic acid salt;
H-Tyr-(D)-Ala-(D)-Phg-Phe-NH₂ trifluoroacetic acid salt;
H-Tyr-D-Arg-Phe-Phe(pF)-NH₂ bis-trifluoroacetic acid salt;
H-Tyr-D-Arg-Phe-D-Phe(pF)-NH₂ ditrifluoroacetic acid salt;
H-Tyr-D-Ala-Phe-Phe(pF)-NH₂ trifluoroacetic acid salt; and
H-Tyr-D-Ala-Phe-D-Phe(pF)-NH₂ trifluoroacetic acid salt.

52. The pharmaceutical composition of claim 51, wherein said peptide is H-Tyr-D-Nva-Phe-Phe-NH₂.

53. The pharmaceutical composition of claim 51, wherein said peptide is H-Tyr-D-Ser-Phe-Phe-NH₂.

54. The pharmaceutical composition of claim 51, wherein said peptide is H-Tyr-D-Arg-Phe-Phe-NH₂.

55. A method for the treatment of pain, comprising the step administering to a mammal in need of such treatment a pharmaceutically effective amount of a peptide selected from the group consisting of:
H-Tyr-Aib-Phe-Phe-NH₂;
H-Tyr-D-Nle-Phe-Phe-NH₂;
H-Tyr-D-Ala-Phe-2′-Nal-NH₂,
H-Tyr-D-Ala-D-Phe-Phe-NH₂;
H-Tyr-D-Ala-Phe(4NO₂)-Phe(4NO₂)-NH₂;
H-Tyr-D-Ala-Phe-Tic-NH₂;
H-Tyr-D-Ala-Phe-Phe(NMe)-NH₂;
H-Tyr-D-Ala-Phe-1′Nal-NH₂;
H-Tyr-D-Ala-Trp-Phe-NH₂;
H-Tyr-D-Ala-Phe-Trp-NH₂;
H-Tyr-VAla-Phe-Phe-NH₂;
VCH₂-Tyr-D-Ala-Phe-Phe-NH₂;
H-Tyr-D-Nle-Phe-Trp-NH₂;
H-Tyr-D-Nle-Phe-2′-Nal-NH₂;
H-Tyr-D-Nle-Trp-Phe-NH₂;
H-Tyr-D-Ala-Trp-2′-Nal-NH₂;
H-Tyr-D-Nle-Trp-2′-Nal-NH₂;
H-Tyr-D-Nle-Trp-Trp-NH₂;
H-Tyr-D-Nva-Phe-Phe-NH₂;
H-Tyr-D-Ser-Phe-Phe-NH₂;
H-Tyr-D-Val-Phe-Phe-NH₂;
H-Tyr-D-Leu-Phe-Phe-NH₂;
H-Tyr-D-Ile-Phe-Phe-NH₂;
H-Tyr-D-Abu-Phe-Phe-NH₂;
H-Tyr-Chl-Phe-Phe-NH₂;
H-Tyr-Cle-Phe-Phe-NH₂;
H-Tyr-D-Arg-Phe-Phe-NH₂;
H-Tyr-D-Cys-Phe-Phe-NH₂;
H-Tyr-D-Thr-Phe-Phe-NH₂;
H-DMT-D-Ser-Phe-Phe-NH₂;
H-Tyr-D-Ala-Phe-Phe-OH trifluoroacetate;
H-Tyr-D-Ala-Phe-Phg-NH₂ trifluoroacetic acid salt;
H-Tyr-D-Arg-Phe-Hph-NH₂ bis-trifluoroacetic acid;
H-DMT-D-Ala-Phe-Phe-NH₂ trifluoroacetic acid;
H-D-DMT-D-Ala-Phe-Phe-NH₂ trifluoroacetic acid salt;
H-Tyr-D-Ala-Phe-Hph-NH₂ trifluoroacetic acid salt;
H-Tyr-D-Ala-Phe-Cys(Bzl)-NH₂ trifluoroacetic acid salt;
H-Tyr-D-Arg-Hph-Phe-NH₂ bis-trifluoroacetic acid salt;
H-Tyr-D-Arg-Phg-Phe-NH₂ bis-trifluoro acetic acid salt;
H-Tyr-D-Ala-Phe-Phe-CH₂OH hydrochloride salt;
H-Tyr-D-Ala-Hph-Phe-NH₂ trifluoroacetic acid salt;
H-Tyr-D-Met-Phe-Phe-NH₂ trifluoroacetic acid salt;
H-Tyr-D-Arg-Phe-D-Phe-NH₂ bis-trifluoroacetic acid salt;
H-Tyr-D-Ala-Phg-Phe-NH₂ trifluoroacetic acid salt;
H-Tyr-(D)-Ala-(D)-Phg-Phe-NH₂ trifluoroacetic acid salt;
H-Tyr-D-Arg-Phe-Phe(pF)-NH₂ bis-trifluoroacetic acid salt;
H-Tyr-D-Arg-Phe-D-Phe(pF)-NH₂ ditrifluoroacetic acid salt;
H-Tyr-D-Ala-Phe-Phe(pF)-NH₂ trifluoroacetic acid salt; and
H-Tyr-D-Ala-Phe-D-Ph(pF)-NH₂ trifluoroacetic acid salt.

56. The method of claim 55, wherein said peptide is H-Tyr-D-Nva-Phe-Phe-NH₂.

57. The method of claim 55, wherein said peptide is H-Tyr-D-Ser-Phe-Phe-NH₂.

58. The method of claim 55, wherein said peptide is H-Tyr-D-Arg-Phe-Phe-NH₂.

59. A compound of formula (B): 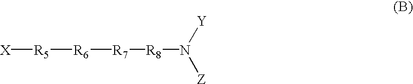
and salts thereof wherein,
a) R₅ is Tyr or 2′,6′-dimethyltyrosine, or an analog or derivative thereof;
b) R₆ is D-Ala or D-Arg;
c) R₇ is Phe(pF);
d) R₈ is Phe or Phe(pF);
e) X is H or C₁₋₆ alkyl; and
f) Y and Z are independently H, aralkyl or C₁₋₆ alkyl.

60. The compound according to claim 59, wherein R₆ is D-Ala.

61. The compound according to claim 59, wherein R₆ is D-Arg.

62. The compound according to claim 59, R₈ is Phe.

63. The compound according to claim 62, wherein R₆ is D-Ala.

64. The compound according to claim 62, wherein R₆ is D-Arg.

65. The compound according to any one of claims 59-64 wherein X is H, and Y and Z are both H.

66. The compound according to claim 59, wherein said compound is selected from the group consisting of:
H-Tyr-D-Ala-Phe(pF)-Phe(pF)—NH₂; and
H-Tyr-D-Ala-Phe(pF)-Phe-NH₂.

67. The compound H-Tyr-D-Ala-Phe(pF)-Phe-NH₂.

68. The compound according to claim 59, wherein said compound is selected from the group consisting of:
H-Tyr-D-Arg-Phe(pF)-Phe(pF)—NH₂ and
H-Tyr-D-Arg-Phe(pF)-Phe-NH₂.

69. The compound H-Tyr-D-Arg-Phe(pF)-Phe-NH₂,

70. A pharmaceutical composition comprising a compound according to any one of claims 59-64, or 66-69 in admixture with a pharmaceutically acceptable carrier.

71. A pharmaceutical composition comprising a compound according to claim 65, in admixture with a pharmaceutically acceptable carrier.

72. A method for the treatment of pain comprising, administering to a mammal in need of such treatment a pharmaceutically effective amount of a compound according to any one of claims 59-64 or 66-69.

73. The method of claim 72, wherein said peptides are administered to a human at a dosage of between 0.05 mg/kg and 20 mg/kg.

74. The method of claim 73, wherein said peptides are administered at a dosage of between 0.1 mg/kg and 1.0 mg/kg.

75. A method for the treatment of pain comprising, administering to a mammal in need of such treatment a pharmaceutically effective amount of a compound according to claim 65.

76. The method of claim 75, wherein said peptides are administered to a human at a dosage of between 0.05 mg/kg and 20 mg/kg.

77. The method of claim 75, wherein said peptides are administered at a dosage of between 0.1 mg/kg and 1.0 mg/kg.
